SEQUENCE LISTING (3 sequences)

SEQ ID NO: 1 - DNA, 38 bp, Synthetic construct
  source 1..38: mol_type = other DNA
cgaccgcgtttcccaagaaagcaagtattggttggtcg

SEQ ID NO: 2 - DNA, 29 bp, Synthetic construct
  source 1..29: mol_type = other DNA
ccggtggtgtagttccggcgtggggaagg

SEQ ID NO: 3 - DNA, 28 bp, Synthetic construct
  source 1..28: mol_type = other DNA
accatctgtgtaaggggtaaggggtggt

(19) United States
(12) Patent Application Publication
(10) Pub. No.: US 2026/0297592 A1
(43) Pub. Date: Oct. 1, 2026

(54) Multiple measurements using an electrochemical aptamer-based sensor

(71) Applicant: THE UNIVERSITY OF MELBOURNE, The University of Melbourne (AU)

(72) Inventors: Alastair George Stewart, The University of Melbourne (AU); Yiling Yang, The University of Melbourne (AU); Ranjith Rajasekharan Unnithan, The University of Melbourne (AU); Xumei Gao, The University of Melbourne (AU); Bryce Jackson Widdicombe, The University of Melbourne (AU)

(21) Appl. No.: 19/496,201

(22) PCT Filed: Jun. 26, 2024
(86) PCT No.: PCT/AU2024/050670

§ 371 Date: Dec. 22, 2025

(30) Foreign Application Priority Data
Jun. 26, 2023 (AU) 2023902007

(51) Int. Cl. C12N 15/115; G01N 27/30; G01N 27/327; G01N 33/53; G01N 33/543; G01N 33/84

(52) U.S. Cl. CPC C12N 15/115; G01N 27/302; G01N 27/3277; G01N 33/5308; G01N 33/5438; G01N 33/84; C12N 2310/16

(57) ABSTRACT

The present disclosure relates methods of using an electrochemical aptamer-based sensor to detect and/or measure at least two parameters of a solution or suspension.

### CROSS-REFERENCE TO RELATED APPLICATION

This application claims priority from Australian provisional application no. 2023902007, the entire contents of which are incorporated herein by reference.

### FIELD OF THE INVENTION

The present disclosure relates to methods of using an electrochemical aptamer-based (EAB) sensor to detect and/or measure at least two parameters of a solution or suspension.

### BACKGROUND OF THE INVENTION

Lab-on-a-chip recently emerged as a new tool for drug research, providing a unique approach for integrating microfluidics and microsensors with the potential to improve our understanding of human biology significantly, allowing for accelerated and cost-effective drug discovery. The pH and amino acid of the physiological environment is an important parameter that influences the behaviour of micro-physiological systems and how a drug formulation, or active ingredients within the formulation, function within a micro-physiological system.

Optical and electrochemical sensors are currently at the forefront of the development of miniaturized lab-on-a-chip sensing technologies. However, a shortcoming is that the optical sensors are often limited by the perturbation of the culture (organisms on the wet sensor surface generate structural-functional deficiencies) from the complex cell culture/tissue environment. For electrical sensors the range of measurements reported is limited.

There is a need for detecting multiple physiological parameters using sensors resistant to fouling due to non-specific adsorption of contaminants that are highly biocompatible, rapid, micro-sized, accurate, and suitable for dynamic micro physiological system monitoring, and which have the potential to be miniaturized in even nanoscale medical devices.

The present invention seeks to address one or more of the shortcomings of the prior art.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present disclosure provides methods of using an electrochemical aptamer-based sensor to detect and/or measure at least two parameters of a solution or suspension. Preferably, the at least two parameters are measured using a single measurement. In a preferred embodiment, the invention relates to methods of using an electrochemical aptamer-based sensor to detect and/or measure pH and a target ligand level in a solution or suspension, such as a biological solution or suspension. In some aspects, the present disclosure provides for a method for measuring the pH and presence of a target ligand in a biological solution or suspension by providing the sensor to a biological solution or suspension, wherein one or more signals in the sensor is proportional to the pH and concentration of target ligand in the biological solution or suspension.

In some aspects, the solution or suspension may be a biological sample such as blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, as well as cell culture supernatants. In some aspects, the solution or suspension may be a solution or suspension comprising an active pharmaceutical ingredient, for example a biological sample comprising an active pharmaceutical ingredient. In other aspects, the solution or suspension may be suitable for use in a bioreactor and comprise a plant culture, a bacterial culture, an insect culture, or a mammalian culture.

The sensor includes an aptamer functionalised working electrode. A surface of the working electrode may be functionalised with one or more aptamers. More particularly, the working electrode is functionalised with one type of aptamer and adapted to detect one type of target ligand. Preferably, the working electrode is functionalised with a plurality of a single type of aptamer. More preferably, a surface of the working electrode is functionalised with a self-assembled monolayer of a single type of aptamer.

The aptamers may be single or double stranded oligonucleotides of DNA, RNA or PNA. In certain aspects, the aptamers are single stranded. In certain aspects, the aptamers are of between about 20 to about 100 nucleic acids in length.

The aptamer binds a target ligand. The target ligand may be selected from an analyte, an amino acid, a peptide, a small protein, a metabolite, a hormone, a steroid, a nucleic acid oligomer, a saccharide, a cofactor, an enzyme, a metal or a carbohydrate. In some aspects, the target ligand may be an active pharmaceutical ingredient.

The aptamer comprises a redox label. Preferably, the aptamer is functionalised with a redox label at the 3′ or 5′ terminus of the aptamer. More preferably, the aptamer is functionalised with a redox label via an amide link at the 3′ terminus of the aptamer. In some aspects, the redox label is selected from methylene blue, ferrocene, viologen, anthraquinone or any other quinones, daunomycin, organo-metallic redox labels, for example porphyrin complexes or crown ether cycles or linear ethers, ruthenium, bis-pyridine, tris-pyridine, bis-imidizole, cytochrome c, plastocyanin, and ethylenetetraacetic acid-metal complexes, or combinations thereof. Preferably, the redox label is methylene blue.

The working electrode may be selected from: gold, a gold-coated metal, aluminium, copper, palladium, titanium, tungsten, silver, platinum, chromium, nickel, carbon (including graphite, nanotubes, graphene), mercury films, an oxide-coated metal, conductive polymers, or any other electrical conductive materials. In a preferred embodiment, the working electrode is gold. In some aspects, the gold working electrode is between about 1 nm and 1 mm thick, including about 10, 20, 30, 40, 50, 60, 70, 80, 90, 100, 200, 300, 400, 500, 600, 700, 800, and 900 μm. In a particularly preferred embodiment, the working electrode has a thickness of about 200 μm. In some aspects, the working electrode has a thickness of between about 1 nm and 10 μm, including about 1, 10, 20, 30, 40, 50, 60, 70, 80, 90, 100, 200, 300, 400, 500, 600, 700, 800, and 900 nm.

In some aspects, the aptamer is affixed to the gold working electrode via a gold-sulfide bond provided by a thiol at the 3′ or 5′ terminus of the aptamer, preferably the 5′ terminus of the aptamer.

In some aspects, the aptamer functionalised working electrode may comprise a polymer coating. The polymer coating may be any suitable anti-fouling polymeric material, including but not limited to agarose.

Electrical sensing between the aptamer and the working electrode may be operably connected to electrical measurement devices, such as a potentiator or voltameter.

In some aspects, the aptamer binds a target ligand, which may cause a conformational change that changes the distance between the redox label and the working electrode. An electrical signal in the sensor may depend on the distance between the redox label and the working electrode.

One or more electrical signals in the sensor may be proportional to the pH and concentration of the target ligand in the solution or suspension. An electrical signal may include: peak potential, faradic current, a non-faradic current, and combinations thereof. Preferably, the pH of the solution or suspension may be measured using a peak potential (V) electrical signal. Preferably, the target ligand may be detected and/or measured using a non-faradic current (A) electrical signal. In a particularly preferred embodiment, the pH and target ligand may be measured using a single measurement, preferably Square-Wave-Voltammetry (SWV).

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Depiction of the structure of an aptamer sequence of the present disclosure. Aptamer sequence 5′ end functionalised with thiol-bond and 3′ end coupled with methylene blue.

FIG. 2. Scheme showing the aptamer target molecule detection on a gold electrode, showing the change in structure of the SAM upon target molecule binding. The gold surface of the working electrode was functionalised with a mono layer of aptamer sequence through thiol-bond binding on the gold electrode surface. The target analyte concentration, aptamer structure, and distance between the redox reporter Methylene Blue and the gold surface change, changed the electron transfer rate.

FIG. 3. a) Schematic showing the connection of the potentiostat to the working electrode (WE), counter electrode (CE) and the reference electrode (RE); b) graph showing the change in peak-potential with high H⁺ concentration (low pH) and low H⁺ concentration (high pH). When the pH shifts high, the peak potential decreases, when the pH shifts low, the peak potential increases.

FIG. 4. Depiction of the potential waveform used in the Square Wave Voltammetry (SWV) measurement by combining a square wave potential and a staircase potential. The SWV applied a potential between WE and RE which is equivalent to a square wave potential plus a staircase potential. Under such way to detect the current signal between CE and WE.

FIG. 5. SWV was performed in “Working Buffer” containing 1 M phenylalanine (top curve, dot line) and without low concentration phenylalanine 10 μM (bottom curve, solid line). The potential is the potential vs Ag/AgCl reference electrode, and the current is the current generated by the reduction or oxidation of some chemical substance at an electrode (faradaic current). Such phenylalanine concentration alters the kinetic transfer efficiency change, which could be measured by the SWV.

FIG. 6. Graph showing the titration measurement results on the aptamer sensor, where the dot is the measured point, and the solid line is the Langmuir-Hill fitted curve.

Phenylalanine in the “Working Buffer” was titrated from 1 nM to 3 mM and the Di (dot) was calculated and then fitted into a Langmuir-Hill fitted curve (solid). Where the solid curve equation is:

Dᵢ=0.014+(1.27−0.014)*Phe^{0.53}/0.00054^{0.53}+Phe^{0.53}(R²=0.996).

FIG. 7. Graph showing the sensor phenylalanine concentration hysteresis measurement. Sensor 1 starts below sensor 2. After gold electrode fabrication and incubation overnight, the sensor was measured under SWV from high phenylalanine concentration to low phenylalanine concentration (Forward, 3 mM-10 μM) and low phenylalanine concentration to high phenylalanine concentration (Backward, 10 μM-3 mM). For each concentration point, 20 consecutive measurements were taken.

FIG. 8. Graph showing the titration curve of a sensor recoated with aptamer. One electrode was recoated three times and titration of phenylalanine form 1 nM to 3 mM was performed. The dark line is the average of the 3 experiments, and the shaded area is the standard deviations.

FIG. 9. Graph showing the repeatability of a titration curve over different electrodes with the same fabrication process. The 6 electrodes were fabricated together and in one solution pod, did the titration of phenylalanine from 1 nM to 3 mM. Where the marker is the measured and calculated Di and the solid line is the hill-fitted curve for each marker group.

FIG. 10. Graph showing the SWV for the aptamer pH sensor performed in a working buffer. The solid curve with the left most peak has a pH of 7.97 and the dot curve with the right most peak has a pH of 6.20.

FIG. 11. Graph showing sensor response curves for the pH-detection in “Working buffer”. The solution “Working Buffer” pH was changed by adding acid or alkaline. The pH was confirmed by commercial pH meter before changing to another pH.

FIG. 12. Graph showing a pH calibration curve at varying pH values. Cross symbol (Forward) was measured from low pH to high pH and dot (backward) was measured from high pH to low pH. Before moving between different pH solutions, the sensor was rinsed with DI water. The LR Backward and the LR Forward are the lines fitted with logistic regression respectively to Backward data and Forward data. Each solution was measured with 20 groups of data under SWV 300 Hz. The dotted line is the linear regression fitted curve on backward data with fitted equation y=−0.0377*x+0.0123 (R²=0.99), and the solid line is the linear regression fitted curve on forward data with fitted equation y=−0.0377*x+0.0130 (R²=0.99).

FIG. 13. Graph showing the pH calibration curve measured in a “Working buffer” and a “Melbourne medium” with varying pH values from 6 to 8. The dot is the Melbourne Medium measured data peak potential data, and the cross is the working buffer measured peak potential data. The dot line represents LR Melbourne Medium with equation y=−0.034*x−0.0280 (R²=0.98), and the solid line represents LR Working buffer with equation y=−0.033x−0.030 (R²=0.99). The assessment of functional compatibility in different types of cell culture medium with identical sensors. After fabrication of gold electrodes, pH calibration curve was measured in “Working Buffer” and “Melbourne Medium” with varying pH values from 6 to 8. The solution was sterilized and prepared in advance with different pH values. Before moving between different pH solutions, the sensor was rinsed with DI water. The LR MM and LR WB are the lines fitted with logistic regression respectively to “Working Buffer” data and “Melbourne Medium” data.

FIG. 14. Graph showing the measured potential of a sensor over time in a solution of varying pH in a “working buffer”. From top to bottom the pH values are 8.07, 7.58, 7.05, 6.59, and 6.27. To evaluate sensor's stability, after sensor's overnight fabrication, 100 successive SWV was measured within a 10-minute period in 5 different pH values which were pre-adjusted in “Working Buffer” with values pH=6.27, pH=6.59, pH=7.05, pH=7.58, pH=8.07. The sensor was rinsed with Di water before every time move it into a new solution. The SWV scan frequency is 300 Hz. Each pH solution was 15 ml in the pot.

FIG. 15. Graph showing the measured potential of a sensor over time in a solution of varying pH in a “Melbourne medium”. From top to bottom the pH values are, 8.00, 7.51, 7.09, 6.50, and 6.23. After sensor's overnight fabrication, 100 successive SWV was measured within a 10-minute period in 5 different pH values which were pre-adjusted in “Melbourne Medium” with value pH=6.23, pH=6.50, pH=7.09, pH=7.51, pH=8.00. The sensor was rinsed with Di water before every time move it into a new solution. The SWV scan frequency is 300 Hz. Each pH solution was 15 ml in the pot.

FIG. 16. Graph showing the potential of a doxorubicin aptamer probe at pH of 7.87, 7.78, 7.61, 7.36, 6.88, and 6.65. To assess the universal detection-pH among all aptamer sequences, electrodes coated with aptamer sequences Doxorubicin aptamer overnight before the measurement (Same fabrication process in section 1). The Doxorubicin aptamer gold electrode was interrogated by 6 different prepared 15 ml “Working Buffer” with pH=7.87,7.78, 7.61, 7.36, 6.88, 6.65. Each solution was measured with 10 groups of data under SWV with a 300 HZ frequency. The dot point is the measured mean of 10 groups of data under each pH solution and the bar is the standard deviation. The solid line is the linear regression fitted curve with equation y=−0.360*x+0.0032 (R²=0.99).

FIG. 17. Graph showing the potential of a L-tryptophan aptamer probe at pH of 7.84, 7.71, 7.56, 7.28, 6.79, and 6.62. To assess the universal detection-pH among all aptamer sequences, electrodes coated with aptamer sequences L-tryptophan aptamer overnight before the measurement (Same fabrication process in section 1). The Doxorubicin aptamer gold electrode was interrogated by 6 different prepared 15 ml “Working Buffer” with pH=7.84,7.71, 7.56, 7.28, 6.79, 6.62. Each solution was measured with 10 groups of data under SWV with a 300 HZ frequency. The dot point is the measured mean of 10 groups of data under each pH solution and the bar is the standard deviation. The solid line is the linear regression fitted curve with equation y=−0.335*x+0.00359 (R²=0.99).

FIG. 18. Diagram showing the configuration of an on-chip sensor lid. The left diagram shows the bottom view with each electrode labelled and the right diagram shows the side view of the on-chip sensor. The cylinder in the middle is the supporter and holder for Ag/AgCl electrode, Au electrode, and Pt electrode to insert in and generate an approximate plane surface. There are two holes located on two sides of the cylinder to insert a tube for nutrition and waste flow. The lid was printed by biocompatible material.

FIG. 19. Diagram showing the on-chip sensor well overview. There are three flutes in the perfusion well plate. One flute is for the nutrition inlet, when the flute liquid level is greater than the second middle-round flutes, the medium will flow in the cell culture flute gradually. When the second middle-round flutes medium exceeds its flute wall, the waste medium will flow out and be ejected into the waste reservoir. The sensor-well lid covers the perfusion well plate with a 1 mm distance between the cells on the bottom well, to precisely measure the cell environment bio information as well as protect the SAM layer on gold electrode surface. The flat sensor lid surface and the small distance between the cell and sensor ensures the laminar flow over the cell surface. The wells were printed by biocompatible material.

FIG. 20. Sensitivity prediction based on measurements in Melbourne Medium and Working Buffer. 1000 measurements in Melbourne Medium and Working Buffer under different pH condition were taken with SWV scan frequency of 300 Hz. Here the mean potential under each pH was calculated and extracted each pH condition's noisy potential out by subtracting the measurement potential with mean potential. After that, all noisy potential signals were fitted into Gaussian Noise distribution model. From the fitted model, the standard deviation (o) and mean (u) of noise potential were calculated. Based on the standard deviation (o) and sensor slope (0.037 mV/pH), predicted the resolution, which is 0.08 pH (three-sigma law, 99.7% prediction accuracy).

FIG. 21. Graph showing the change in Di of the sensor measuring more than 2 analytes (phenylalanine and L-tryptophan titrated in that order) at same time. To confirm the sensor can measure more than 2 analytes at same time, two experiments have been conducted. In experiment 1 settings, two electrodes covered with phenylalanine aptamer and another two electrodes covered with L-tryptophan aptamer were fabricated and incubated overnight. All four electrodes are tested in the same condition at the same time. In the titration process, the phenylalanine (from 1 nM to 3 mM) in “Working Buffer” was first titrated, and then the L-tryptophan (from 1 nM to 3 mM) in “Working Buffer” was titrated. The cross in the plot indicate the phenylalanine sensor measurements results and dot in the plot indicate L-tryptophan sensor measurement results.

FIG. 22. Graph showing the change in peak potential of the sensor measuring more than 2 analytes (phenylalanine and L-tryptophan titrated in that order) at same time. After titration process in FIG. 21's process, sensors were taken into working buffer under different pH values (6.61, 7.05, 7.31, 7.54, 7.81), in each pH, 10 measurements were taken under 300 Hz. The left subplot is the two phenylalanine electrode pH response curve and its linear regression fitted curve. The right subplot is the two L-tryptophan electrode pH response curve and its linear regression fitted curve. The bar is the standard deviation.

FIG. 23. Graph showing the change in Di of the sensor measuring more than 2 analytes (L-tryptophan and phenylalanine titrated in that order) at same time. In experiment settings, two electrodes covered with phenylalanine aptamer and another two electrodes covered with L-tryptophan aptamer were fabricated and incubated overnight. All four electrodes are tested in the same condition at the same time. In the titration process, the L-tryptophan (from 1 nM to 3 mM) in “Working Buffer” was first titrated, then the phenylalanine (from 1 nM to 3 mM) in “Working Buffer” was titrated. The cross in the plot indicate the phenylalanine sensor measurements results and dot in the plot indicate L-tryptophan sensor measurement results.

FIG. 24. Graph showing the change in peak potential of the sensor measuring more than 2 analytes (L-tryptophan and phenylalanine titrated in that order) at same time. After titration process in FIG. 23's process, sensors were taken into working buffer under different pH values (6.81, 6.99, 7.21, 7.38, 7.54), in each pH, 10 measurements were taken under 300 Hz. The left subplot is the two phenylalanine electrode pH response curve and its linear regression fitted curve. The right subplot is the two L-tryptophan electrode pH response curve and its linear regression fitted curve. The bar is the standard deviation.

FIG. 25. pH measurement stability under different flowrate conditions. To integrate the sensor into dynamic in vitro system, the inventors confirmed whether flowrate influences the measurement. They designed an experiment which allow the sensor in the on-chip devices to take 10 groups of measurement with SWV scan frequency 300 Hz in pH=7.3 working buffer under 6 different flowrates: 0 μl/min, 0.5 μl/min, 1 μl/min, 2 μl/min, 4 μl/min, 8 μl/min. The flowrate was achieved and controlled by microfluidic pump and varied from low to high and high to low accordingly. The sensor gold electrode was fabricated and incubated overnight before the measurements. In the left subplot, the diamond is the mean of the data measured with flowrate change from low to high, and the solid is the linear regression fitted curve for those data points (Forward). The cross is the mean of the data measured with flowrate change from high to low on each flowrate and the dash line is the linear regression fitted curve for those data points (Backward). The bar is the standard deviation. In the right subplot, the pH was calculated based on previous calibrated curve. Where the diamond is the calculated pH based on Forward measurements, and the cross is the calculated pH based on Backward measurements. The bar is the standard deviation.

FIG. 26. Dynamic flow condition test on Phe and L-Tryp aptamer sensor experiment 1. And for the target analyte stability under dynamic condition, Phe and L-Tryp aptamer coated electrodes were placed in 24-cell well, start with static condition and then increased to 0.5 μl/min, 1 μl min, 2 μl/min, 4 μl/min, and 8 μl/min. In this experiment, two concentrations of target analytes were tested separately (0 μM and 100 μM, on both L-tryptophan and phenylalanine). Under each concentration's flowrate, 5 groups of measurements were taken with SWV=300 Hz. In left subplot, the solid line is L-tryp aptamer reading and dot line is Phe aptamer sensor reading. In right subplot, the solid line is L-tryp sensor reading obtained passing 100 μM L-tryptophan concentration Working Buffer, and the dash-line is the Phe sensor reading obtained passing 100 μM phenylalanine concentration Working Buffer.

FIG. 27. Dynamic flow condition test on PHE and L-TRP aptamer sensor experiment 2. And for the target analyte stability under dynamic condition, Phe and L-Tryp aptamer coated electrodes were placed in 24-cell well, start with 8 μl/min and then decreased to 4 μl/min, 2 μl min, 1 μl/min, 0.5 μl/min, and static conditions. In this experiment, two concentrations of target analytes were tested separately (0 μM and 100 μM, on both L-tryptophan and phenylalanine). Under each concentration's flowrate, 5 groups of measurements were taken with SWV=300 Hz. In left subplot, the solid line is L-tryp aptamer reading and dot line is Phe aptamer sensor reading. In right subplot, the solid line is L-tryp sensor reading obtained passing 100 μM L-tryptophan concentration Working Buffer, and the dash-line is the Phe sensor reading obtained passing 100 μM phenylalanine concentration Working Buffer.

FIG. 28. Aptamer sensor flowrate hysteresis experiment, part 1. For the flowrate hysteresis test on target analyte. Phe aptamer electrode was placed into 100 μM phenylalanine Working Buffer and L-Tryp aptamer electrode was placed into 100 μM L-tryptophan Working Buffer. Flowrate changes from “Forward” (flowrate change from low to high) to “Backward” (flowrate change from high to low). Under each flower rate, 5 groups of measurements were taken. The left subplot is the phenylalanine electrode dynamic response in 100 μM phenylalanine Working Buffer, dash-line is forward and solid line is backward. The right subplot is the L-tryptophan electrode dynamic response in 100 μM L-tryptophan Working Buffer, dot-line is forward and solid line is backward.

FIG. 29. Aptamer sensor flowrate hysteresis experiment, part 2. For the flowrate hysteresis test on target analyte. Phe aptamer electrode was placed into 100 μM phenylalanine Working Buffer and L-Tryp aptamer electrode was placed into 100 μM L-tryptophan Working Buffer. Flowrate changes from “Backward” (flowrate change from high to low) to “Forward” (flowrate change from low to high). Under each flower rate, 5 groups of measurements were taken. The left subplot is the phenylalanine electrode dynamic response in 100 μM phenylalanine Working Buffer, dash-line is backward and solid line is forward. The right subplot is the L-tryptophan electrode dynamic response in 100 μM L-tryptophan Working Buffer, dot-line is backward and solid line is forward.

FIG. 30. Pictorial representation of the leak-less reference electrode. Before the measurement start, leakless reference electrode was fabricated. The torch seals the one end of capillary glass tube with platinum wire. Then filled with 3M NaCl and inserted with electrochemical coated Ag/AgCl wire into it. The other end with Ag/AgCl wire was sealed by parafilm, and the exposed Ag wire was covered with copper tape (Walker NL, 2021).

FIG. 31. Open circuit potential (OCP) measurement of the leak-less reference electrode in 3M NaCl against a commercial reference electrode. Open circuit potential measurement was taken in 3M NaCl for the leakless electrode against commercial reference electrode (BASMF2052). And to compare the stability, another commercial reference electrode (BASMF2052) was also taken open circuit potential measurement against the same reference electrode. The dot line in the results is the OCP results for commercial reference electrode and the solid line is the OCP results for the leakless reference electrode.

FIG. 32. Cell metabolism measurement timeline. Five days measurement was taken to perform a cell metabolism measurement. The cell line is A549 lung cancer cell, medium is Melbourne Medium under static condition in 24 well plate. In day 0, 3 electrodes were fabricated and start cell seeding to insure fully confluency before the metabolism measurement. And all the components were sterilized by UV and ethanol. 2 wells were seeded with cell, on for cell metabolism measurement by the sensor another for the control group. And in Day 1, Day 2 and Day3, the sensor did calibration in incubator (37 Celsius, 5% CO2) include phenylalanine calibration with 0 μM, 10 μM and 100 μM with scan frequency 1000 Hz and 30 Hz. In Day 1, Day 2 and Day3, after calibration, 5 hours continuous reading with 15 minutes interval was taken. In In Day 1, Day 2, Day3 and Day4, cell microscopy was taken to ensure the cell has no contamination.

FIG. 33. Cell microscopy results based on cell metabolism measurement. Cell microscopy results from Day 1 to Day 4 was listed in the figure from left to right. There has neither obvious morphology nor cell viability between the cell well with sensor (top) and without sensor (bottom).

FIG. 34. Cell metabolism measurement results. The left subplot is the measurements results of phenylalanine and the right subplot is the measurement results of pH. Where the shaded area is the human plasma physiological range. From the results of the measurement on phenylalanine, in the first day first 5 hours, the cell culture can stay closely stay in physiological range to maintain cell grow and divide. In day 2, the phenylalanine level already down to 1 μM in hypo physiological range, as cell growth and divide need the amino acid consumption. From the results measurement of the pH, As cells consume nutrients from the culture medium, metabolic by-products and waste are generated, including organic acids, lactic acid. If the rate of nutrient consumption exceeds the supply of fresh medium, the accumulation of metabolic by-products can lower the ph. From this results, the sensor's ability to reflect the cell metabolism has been ascertained and which further confirm that Regular monitoring of nutrient levels, pH, and waste accumulation is important to ensure optimal cell growth and maintain a healthy cell culture in long time experiment to improve prediction efficacy.

FIG. 35. pH real-time measurement set up overview.

FIG. 36. Real-time measurement of pH with syringe pump.

FIG. 37. Real-time measurement of pH with pump after Savitzky-Golay filtering.

FIG. 38. Phenylalanine real-time measurement set up overview.

FIG. 39. Real-time measurement of phenylalanine with pump.

FIG. 40. pH sensing temperature dependence. The black line represents measurements taken at ambient temperature and grey line represents measurements taken at 37 Celsius. The solid lines are linear regression fitted curves.

FIG. 41. Relative signal change under different frequencies in Melbourne Medium. The error bars are the maximum and minimum values among 5 scans.

FIG. 42. Phenylalanine-relative signal change under 1000 Hz and 10 Hz in human plasma like conditions. Black represents the signal-off frequency response, grey represents the signal-on frequency response. The error bars are the maximum and minimum values among 3 independent experiments.

FIG. 43. Phenylalanine-Di ratio response curve. The error bars are the maximum and minimum values among 3 independent experiments.

FIG. 44. Leakless reference electrode stability over time. The upper line in grey represents open circuit measurement on day 1, the lower line in black represents open circuit measurement on day 60.

FIG. 45. Leakless reference electrode potential & commercial reference electrode potential differences before & after ethanol spray. The plot includes: Leakless reference electrode potential before spray (Leakless before); Leakless reference electrode potential after spray (Leakless after); Commercial reference electrode potential before spray (Commercial before); Commercial reference electrode potential after spray (Commercial after).

FIG. 46. Schematic of electrode holder and three electrodes in 24-well culture plate for cell culture monitoring. 1, Parafilm; 2, Gold; 3, Glass; 4, Platinum; 5, AgCl; 6, 3M NaCl; 7, Silver; 8, PTFE.

FIG. 47. Experiment steps overview of cell culture real-time monitoring by EAB sensor.

FIG. 48. EAB sensor pH real-time measurement results in cell culture. The error bar is the maximum and minimum value. (n=3).

FIG. 49. pH measurement benchmarked by pH stripes.

FIG. 50. EAB sensor phenylalanine real-time measurement results in cell culture. The error bar is the maximum and minimum value. (n=3).

FIG. 51. Benchmark phenylalanine measurement by GC-MS.

FIG. 52. Schematic showing agarose coating of WE.

FIG. 53. EAB sensor with agarose coating response curve.

FIG. 54. Multiplexed EAB sensor array system design overview.

FIG. 55. PCB board design for multiplexed sensor array system.

FIG. 56. EAB sensor electrode array PCB design.

FIG. 57. Leakless reference electrode version 2.

FIG. 58. 3D printed multiplexed EAB sensor array lid design.

FIG. 59. Fabricated and assembled multiplexed EAB sensor array system.

FIG. 60. SWV scan comparison of gold as counter electrode and platinum as counter electrode.

FIG. 61. Leakless reference electrode V2 potential stability. The plot includes: the measurement of leakless reference electrode version 1 (glass tube); and the measurement of leakless reference electrode version 2 (plastic tube).

### DETAILED DESCRIPTION OF THE EMBODIMENTS

The present disclosure provides methods of using an electrochemical aptamer-based sensor to detect and/or measure at least two parameters of a solution or suspension. Preferably, the at least two parameters are measured using a single measurement. In a preferred embodiment, the invention relates to methods of using an electrochemical aptamer-based sensor to detect and/or measure pH and a target ligand level in a solution or suspension, such as a biological solution. In some aspects, the present disclosure provides for a method for measuring the pH and presence of a target ligand in a biological solution or suspension by providing the sensor to a biological solution or suspension, wherein one or more signals in the sensor is proportional to the pH and concentration of target ligand in the biological solution or suspension.

In some aspects, the solution or suspension may be a biological sample such as blood, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, as well as cell culture supernatants. In some aspects, the solution may be a solution comprising an active pharmaceutical ingredient, for example a biological sample comprising an active pharmaceutical ingredient. In other aspects, the solution or suspension may be suitable for use in a bioreactor and comprise a plant culture, a bacterial culture, an insect culture or a mammalian culture.

The present disclosure concerns electrochemical aptamer-based sensors on electrode interfaces that render the sensors resistant to fouling due to non-specific adsorption of contaminants. The present disclosure concerns using a sensor to detect both pH and a target ligand in complex media, including biological and environmental media, preferably using a single measurement.

In any aspect, the solution or suspension is a turbid solution or suspension. In any embodiment, the turbid solution or suspension may have Nephelometric Turbidity Units (NTU) of equal to or greater than 10 NTU, equal to or greater than 15 NTU, equal to or greater than 20 NTU, equal to or greater than 25 NTU, equal to or greater than 30 NTU, equal to or greater than 35 NTU, equal to or greater than 40 NTU, equal to or greater than 45 NTU, equal to or greater than 50 NTU, equal to or greater than 55 NTU, equal to or greater than 60 NTU, equal to or greater than 65 NTU, equal to or greater than 70 NTU, equal to or greater than 75 NTU, equal to or greater than 80 NTU, equal to or greater than 85 NTU, equal to or greater than 90 NTU, equal to or greater than 95 NTU, equal to or greater than 100 NTU, equal to or greater than 150 NTU, equal to or greater than 200 NTU, equal to or greater than 250 NTU, equal to or greater than 300 NTU, equal to or greater than 350 NTU, equal to or greater than 400 NTU, equal to or greater than 450 NTU, equal to or greater than 500 NTU, equal to or greater than 550 NTU, equal to or greater than 600 NTU, equal to or greater than 650 NTU, equal to or greater than 700 NTU, equal to or greater than 750 NTU, equal to or greater than 800 NTU, equal to or greater than 850 NTU, equal to or greater than 900 NTU, equal to or greater than 950 NTU, equal to or greater than 1,000 NTU, equal to or greater than 1,500 NTU, equal to or greater than 2,000 NTU, equal to or greater than 2,500 NTU, equal to or greater than 3,000 NTU, equal to or greater than 3,500 NTU, equal to or greater than 4,000 NTU, equal to or greater than 4,500 NTU, equal to or greater than 5,000 NTU, equal to or greater than 5,500 NTU, equal to or greater than 6,000 NTU, equal to or greater than 6,500 NTU, equal to or greater than 7,000 NTU, equal to or greater than 7,500 NTU, equal to or greater than 8,000 NTU, equal to or greater than 8,500 NTU, equal to or greater than 9,000 NTU, equal to or greater than 9,500 NTU, or equal to or greater than 10,000 NTU. In any embodiment, the turbid solution or suspension may have NTU of 10 NTU to 100 NTU, 10 NTU to 90 NTU, 10 NTU to 80 NTU, 10 NTU to 70 NTU, 10 NTU to 60 NTU, 10 NTU to 50 NTU, 10 NTU to 40 NTU, 10 NTU to 30 NTU, 10 NTU to 20 NTU, 20 NTU to 100 NTU, 30 NTU to 100 NTU, 40 NTU to 100 NTU, 50 NTU to 100 NTU, 60 NTU to 100 NTU, 70 NTU to 100 NTU, 80 NTU to 100 NTU, or 90 NTU to 100 NTU. In any embodiment, the turbid solution may have a maximum NTU of 10,000 NTU, 9,500 NTU, 9,000 NTU, 8,500 NTU, 8,000 NTU, 7,500 NTU, 7,000 NTU, 6,500 NTU, 6,000 NTU, 5,500 NTU, 5,000 NTU, 4,500 NTU, 4,000 NTU, 3,500 NTU, 3,000 NTU, 2,500 NTU, 2,000 NTU, 1,500 NTU, 1000 NTU, 950 NTU, 900 NTU, 850 NTU, 800 NTU, 750 NTU, 700 NTU, 650 NTU, 600 NTU, 550 NTU, 500 NTU, 450 NTU, 400 NTU, 350 NTU, 300 NTU, 250 NTU, 200 NTU, 150 NTU, 100 NTU or 50 NTU.

The present disclosure concerns a sensor including an aptamer functionalised working electrode. The aptamer functionalised working electrode provides an interface between a sensor body and the surrounding medium, such as a biological medium.

The working electrode is a material capable of transmitting or conducting an electrochemical signal, such as a signal generated by a conformational change in the aptamer due to target binding and/or a signal generated by a change in oxidation state of a redox label.

The working electrode may be selected from: gold, a gold-coated metal, aluminium, copper, palladium, titanium, tungsten, silver, platinum, chromium, nickel, carbon (including graphite, nanotubes, graphene), mercury films, or an oxide-coated metal, conductive polymers, or any other electrical conductive materials. In a preferred embodiment, the working electrode is gold. In some aspects, the gold working electrode is between about 1 nm and 1 μm thick. In some aspects, the working electrode is a gold working electrode with aptamers residing on a surface of the working electrode.

The working electrode may be in the form of any required or desired shape or size. For example, the working electrode may be a wire, planar, or porous.

In some aspects, the gold employed in the gold working electrode disclosed herein is of a certain level of purity or karat. In some aspects, the karat of the gold is of between 7 and 24, including 8, 9, 10, 11, 12, 13, 14, 15, 16, 17, 18, 19, 20, 21, 22, and 23. In some aspects the gold is 9 karat. In other aspects, the gold is of between 9 and 18 karat.

In some aspects, the working electrode has a thickness of between 1 nm and 1 mm, including about 10, 20, 30, 40, 50, 60, 70, 80, 90, 100, 200, 300, 400, 500, 600, 700, 800, and 900 μm. In a particularly preferred embodiment, the working electrode has a thickness of about 200 μm. In some aspects, the working electrode has a thickness of between 1 nm and 10 μm, including about 1, 10, 20, 30, 40, 50, 60, 70, 80, 90, 100, 200, 300, 400, 500, 600, 700, 800, and 900 nm.

The aptamer functionalised working electrode may comprise a polymer coating. The polymer coating may be any suitable anti-fouling polymeric material, including but not limited to agarose. A surface of the working electrode may be functionalised with one or more aptamers. More particularly, the working electrode is functionalised with one type of aptamer and adapted to detect one type of target ligand. Preferably, the working electrode is functionalised with a plurality of a single type of aptamer. Preferably, a surface of the working electrode is functionalised with a self-assembled monolayer of a single type of aptamer.

Aptamers are known in the art and may be specific for almost any target, for example being generated by systematic evolution of ligands by exponential enrichment (see, e.g., Wu et al., Anal. Chem. 91:15335-15344, 2019). Aptamers may include a sequence or string of oligonucleotides, such as DNA aptamers, RNA aptamers, and aptamers comprising non-natural nucleic acids may be used, as well as hybrids of the foregoing and including polymers, such as PNA. In some aspects, the oligonucleotides may be capable of forming “stem-loop” or “hairpin” structures (also referred to “stem-loop” or “hairpin”, or simply “stem-loops” or “hairpins”), with an electroactive label to detect hybridization events. Typical aptamers are about 20-100 bases in length, including 20, 21, 22, 23, 24, 25, 26 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 mers. However aptamers of any size may also be used. Extant aptamers known in the art include those capable of binding target species such as an amino acid, a chemotherapeutic agent, ATP (Zuo, X et al, JACS, 2009, 131 (20), 6944-6945), lysozyme, thrombin (Xiao, Y et al, JACS, 2005, 127 (51), 17990-17991), HIV, trans-acting responsive element, hemin, interferon, vascular endothelial growth factor, prostate specific antigen, dopamine, methotrexate (Flatebo, C et al, ACS Sensors, 2023, 8(1), 150-157), kanamycin (Ferguson, B et al, Sci Transl Med, 2013), platelet-derived growth factor (Lai, R et al, Analytical Chemistry, 2007, 79(1), 229-233), aminoglycoside antibiotics (Rowe, A et al, Analytical Chemistry, 2010, 82(17), 7090-7095), and cocaine (Baker, B. et al, JACS, 2006, 128(10), 3138-3139).

In the present specification, the term “amino acid” refers to a molecule containing both an amino group and a carboxy group. For example, in an α-amino acid, there is an “α-amino group” attached directly to the carbon atom bearing both an amino and a carboxyl group and an “α-carboxyl group” attached directly to the carbon atom bearing both an amino and a carboxyl group. The term “carboxyl” may refer to either a —COOH group or a —COO— group. α-amino acids are of the general form H₂N—CHR—COOH, where R is a side chain or H. The side chain in general is an alkyl chain, which is optionally substituted, commonly but not necessarily at its distal end. The N terminus of the amino acid (or of a peptide) is that end at which the amine functionality (optionally ionised or substituted/protected) is located, and the C terminus is the end at which the carboxyl functionality (optionally ionised or substituted/protected) is located. A one-letter abbreviation system is frequently applied to designate the identities of the twenty “canonical” or proteogenic amino acid residues generally incorporated into naturally occurring peptides and proteins (Table 1). Such one-letter abbreviations are entirely interchangeable in meaning with three-letter abbreviations, or non-abbreviated amino acid names. Non-canonical or non-proteogenic amino acid residues include amino acid residues in D- or L-form that are not among the 20 canonical amino acids generally incorporated into naturally occurring proteins, for example, β-amino acids, homoamino acids, cyclic amino acids, seleno amino acids, thio amino acids, and amino acids with derivatized side chains.

TABLE 1
Three and one-letter abbreviations for the twenty
canonical/naturally-occurring amino acids

| | Three-letter | One-letter |
|---|---|---|
| Amino acid | abbreviation | abbreviation |
| Alanine | Ala | A |
| Glutamine | Gln | Q |
| Leucine | Leu | L |
| Serine | Ser | S |
| Arginine | Arg | R |
| Glutamic Acid | Glu | E |
| Lysine | Lys | K |
| Threonine | Thr | T |
| Asparagine | Asn | N |
| Glycine | Gly | G |
| Methionine | Met | M |
| Tryptophan | Trp | W |
| Aspartic Acid | Asp | D |
| Histidine | His | H |
| Phenylalanine | Phe | F |
| Tyrosine | Tyr | Y |
| Cysteine | Cys | C |
| Isoleucine | Ile | I |
| Proline | Pro | P |
| Valine | Val | V |

Preferably, the aptamer is capable of binding target species such as amino acids or chemotherapeutic agents. Any suitable chemotherapeutic agent may be used. A particularly preferred chemotherapeutic agent includes doxorubicin. More preferably, the aptamer is capable of binding Phe, Tyr or doxorubicin.

In one embodiment, the aptamer capable of binding Phe comprises the sequence

5′-CGACC-GCGTT-TCCCA-AGAAA-GCAAG-TATTG-
GTTGG-TCG-3′.

In one embodiment, the aptamer capable of binding Trp comprises the sequence

5′-CCGGT-GGTGT-AGTTC-CGGCG-TGGGG-AAGG-3′.

In one embodiment, the aptamer capable of binding doxorubicin comprises the sequence 5′-ACCAT-CTGTG-TAAGG-GGTAA-GGGGT-GGT-3′.

The aptamers may be single or double stranded oligonucleotides of DNA, RNA or PNA. In certain aspects, the aptamers are single stranded. In certain aspects, the aptamers are of between about 20 to about 100 nucleic acids in length.

In some aspects the aptamer is affixed to the working electrode through a covalent interaction. In some aspects, the aptamer is bound through a thiol conjugation, alkanethiol, cyclic disulfide, dithiothreitol, diothiols, adenosine or phosphorothioated adenosines. In certain aspects, the aptamer is a single-stranded oligomer, such as ssDNA or RNA, functionalised at the 3′ or 5′ terminus with a thiol functional group, preferably the 5′ terminus. The thiol can react with the gold of the working electrode and provide a gold-sulfide bond to affix aptamers to the working electrode. (See, generally, Odeh et al., Molecules 25(1):3(2020). Martinez-Jothar et al., J. Control Release 28:101-09 (2018), and Danesh et al., Int. J. Pharm. 489:311-17, (2015)). In one aspect, an aptamer solution can be pre-treated with a phosphine, such as with tris-(2-carboxyethyl) phosphine or dithiothreitol, for a period of time between about 30 mins to 5 hours, including about 45, 60, 75, 90, 105, 120, 135, 150, 165, 180, 195, 210, 225, 240, 255, 270, and 285 mins, in order to break any disulfide bonds. While some of the single-stranded oligomer aptamers may be physisorbed to the gold surface, a vast majority of the aptamers on the surface are chemisorbed and anchored through a gold-sulfide bond.

The aptamer binds a target ligand. The target ligand may be selected from an analyte, an amino acid, a peptide, a small protein, a metabolite, a hormone, a steroid, a nucleic acid oligomer, a saccharide, a cofactor, an enzyme, a metal or a carbohydrate. In some aspects, the target ligand may be an active pharmaceutical ingredient.

The structures of some aptamers for known ligands are known in the art, while further aptamers for other ligands can be readily identified though known methods in the art including systematic evolution of ligands by exponential enrichment (SELEX). In some aspects, the aptamer may be an amino acid-binding aptamer, aminoglycoside-binding aptamer, a cocaine-binding aptamer, any small molecule-binding aptamer, a thrombin-binding aptamer, a platelet derived growth factor-binding aptamer, a neuropeptide Y-binding aptamer, any protein-binding aptamer, an inorganic ion-binding aptamer or a DNAzyme binding aptamer.

In some aspects, the target ligand may be any inorganic or organic molecule, for example: a small molecule drug, a metabolite, a hormone, an amino acid, a peptide, a protein, a carbohydrate, a nucleic acid, an analyte, a steroid, a nucleic acid oligomer, a saccharide, a cofactor, an enzyme, a metal, or any other composition of matter. The target ligand may be a therapeutic drug, an antibiotic, an illicit drug, an antibiotic agent, or a chemotherapeutic drug. The target ligand may include a naturally-occurring factor, such as a hormone, metabolite, growth factor, neurotransmitter, or similar. The target ligand may be any other species of interest, for example, species such as pathogens (including pathogen induced or derived factors), toxins, nutrients, and pollutants.

In some aspects of the invention, the aptamer features a terminal redox label. Preferably, the aptamer is functionalised with a redox label at the 3′ or 5′ terminus of the aptamer. More preferably, the aptamer is functionalised with a redox label via an amide link at the 3′ terminus of the aptamer. Preferably, the aptamer is a single stranded oligomer, such as ssDNA or RNA, comprising a redox label at the 3′ terminus. A redox label can be provided as an ester which can react with the amine at the 3′ terminus to link the two together. In some aspects, the redox label is a methylene blue succinimide ester connected to the amine-terminated ssDNA or RNA. In other aspects, the redox label may comprise a porphyrin-containing redox sensor capable of detecting and/or measuring physiological gases such as oxygen, NO and CO.

In some aspects, the terminal label may be of a redox material, such that a change in the distance between the label and the gold working electrode causes a detectable change in electron transfer between the two. As such, when a ligand binds to the aptamer, the physical shape changes and the redox label distance changes, causing a detectable change in electron transfer and the resistance created between the two.

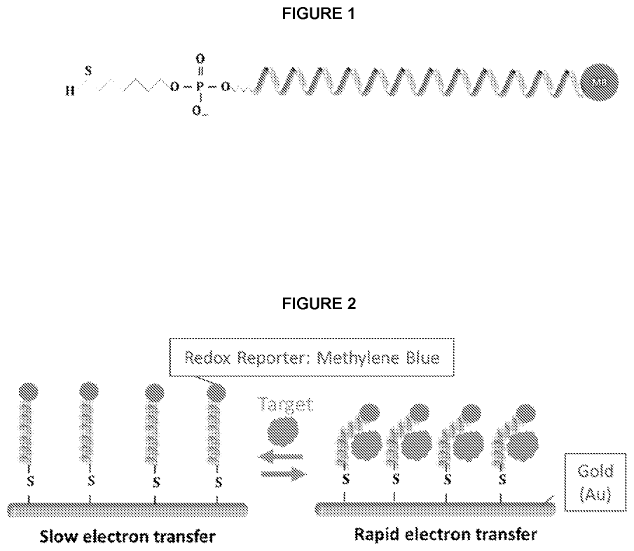

In some aspects, the redox label is capable of electron transfer to or from the electrode, optionally with direct transfer to the working electrode and then to the sensor body. With sufficient proximity and accessibility of a redox label to the electrode, an electrical signal, e.g. current, voltage, or other measurable electrical interaction, will occur between the redox label and the electrode. The redox label may be positioned such that binding of the target ligand to the aptamer causes a measurable change in the electrical signal generated by the redox label. In some aspects, the redox label is positioned at the terminus of the aptamer, for example as depicted in FIG. 1. In other aspects, the redox label is present on a separate polynucleotide strand that binds to the aptamer in the absence of target species and that is displaced by binding of the target species to the aptamer (see, Xiao et al., J. Am. Chem. Soc., 127:17990-91 (2005)). In some aspects, the redox labels may be configured for “turn-off” wherein the redox signal is decreased by the binding of the target ligand. In other aspects, the redox labels may be configured for “turn-on” signalling wherein the redox signal is increased by the binding of the target ligand. The placement of such sensing label can be selected using known methods of designing electrochemical sensors.

In some aspects, the redox label is methylene blue. In other aspects, the redox sensor is selected from known redox labels including methylene blue, ferrocene, viologen, anthraquinone or any other quinones, daunomycin, organo-metallic redox labels, for example porphyrin complexes or crown ether cycles or linear ethers, ruthenium, bis-pyridine, tris-pyridine, bis-imidizole, cytochrome c, plastocyanin, and ethylenetetraacetic acid-metal complexes, or combinations thereof. Preferably, the redox label is methylene blue.

In some aspects, the redox label is linked to the aptamer and then the aptamer-label unit is affixed to the working electrode, such as with a carbon-chain linker or directly with a thymine. In some aspects, the aptamers affix to the working electrode by incubation with an aptamer solution at a concentration of between about 100 nM and 500 nM, including about 200 nm, 300 nm and 400 nm, for a period of between about 1 and 5 hours, including about 2, 3 and 4 hours. In further aspects, the incubation can occur at a temperature suitable for incubation of a plant culture, a bacterial culture, an insect culture, or a mammalian culture such as between about 0 and 100° C., including any range or value therein. Preferably, the incubation can occur at a temperature of between about 20 and 45° C., including about 21, 22, 23, 24, 25, 26, 27, 28, 29, 30, 31, 32, 33, 34, 35, 36, 37, 38, 39, 40, 41, 42, and 43° C.

The target ligand binds the aptamer and the aptamer assumes a conformation such that the redox label is in proximity to the sensor body and/or working electrode, causing the flow or Faradic current or other measurable electronic interactions. In a sensor comprising a plurality of recognition elements, the bulk dynamics of target binding and dissociation and the resulting electronic interactions with the substrate create a measurable electronic signal that is proportional to the concentration of the target species in the sample solution or suspension.

One or more electrical signals in the sensor may be proportional to the pH and concentration of the target ligand in the solution or suspension. An electrical signal may include: peak potential, faradic current, a non-faradic current, and a combination thereof.

Preferably, the pH of the solution or suspension may be measured using a peak potential (V) electrical signal. In a preferred embodiment, the electrochemical aptamer-based sensor is capable of measuring the pH of a solution or suspension within physiological pH range. Preferably, the pH is between about 4 to about 8, including any value or range therein, including about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0.

Preferably, the target ligand may be detected and/or measured using a faradic current (A) electrical signal. In a preferred embodiment, the electrochemical aptamer-based sensor is capable of measuring the concentration of a target ligand in a solution or suspension within physiological ranges. Preferably, the target ligand concentration is between about 0 M to about 10 M, including any range or value therein. Preferably, the target ligand concentration is between about 0 mM to about 100 mM, including any range or value therein, including but not limited to about 0 mM, about 10 mM, about 20 mM, about 30 mM, about 40 mM, about 50 mM, about 60 mM, about 70 mM, about 80 mM, about 90 mM and about 100 mM. Preferably, the target ligand concentration is between about 0 mM and about 10 mM, including any range or value therein, including but not limited to about 0 mM, about 1 mM, about 2 mM, about 3 mM, about 4 mM, about 5 mM, about 6 mM, about 7 mM, about 8 mM, about 9 mM and about 10 mM. Preferably, the target ligand concentration is between about 0 μM and 100 μM, including any range or value therein, including but not limited to about 0 μM, about 10 μM, about 20 μM, about 30 μM, about 40 μM, about 50 μM, about 60 μM, about 70 μM, about 80 μM, about 90 UM, and about 100 μM.

In a particularly preferred embodiment, the pH and target ligand may be measured using a single measurement, preferably Square-Wave-Voltammetry (SWV).

In some aspects, the working electrode is further functionalised with carbon chain materials to prevent non-specific binding and/or to provide a buffer between the redox label and the electrode/sensor body. In some aspects, the carbon chain comprises 6-mercapto-1-hexanol or 6-aminohexanol. The carbon-chains can be bound by incubation of a concentration of between about 100 nM and 500 nm, including about 200, 300 and 400 nm for a period of between about 30 mins and 2 hrs, including 45, 60, 75, 90, and 105 mins. The bound carbon chains may prevent non-specific adsorption of the aptamers onto the electrode surface, thus providing for more reliable signal measurements.

The working electrode can be submerged or contacted with a biological fluid and changes in the electrical current detected. As depicted in FIG. 1, the presence of the aptamers on a surface of the working electrode allows for small ligands to freely associate.

The aptamer functionalised working electrode may be affixed to a sensor body. In some aspects, the sensor body is of an electro-conductive material, such that any electrical signal generated by the aptamer can conduct through the sensor body and to a measuring or detecting device connected thereto. The sensor body may be of any suitable conducting material for electrochemical sensing, including, for example: gold or any gold-coated metal or material, aluminium, copper, palladium, titanium, tungsten, silver, platinum, carbon (including graphite, nanotubes, graphene), mercury films, oxide-coated metals, semiconductor materials, and any other conductive material.

The sensor body may be in the form of any required or desired shape or size. For example, the sensor body may be a disc, cylinder, wire, sphere, paddle, rectangle, strip, array, screen printed or other. Those skilled in the art will appreciate that a narrow sensor body, such as a thin wire, may in some aspects provide further less invasive advantages to in vivo applications.

In some aspects, the sensor body is in electrical communication with a measuring or detecting device, as well as ancillary components such as power supplies or connectors thereto. Such communication may include wires or other electrically conductive elements that connect the sensor body to detectors, measuring devices, controllers, power supplies, voltage regulators, and other control elements which operate the sensing element. The sensor body may further be connected to structures designed and arranged to house or support the sensor body during operation to retain such in place to ensure proper operation.

In some aspects, the sensor body is part of an overall sensor electrode sensing system to detect and/or measure the presence of pH and a target ligand or molecule in a medium, such as a biological medium. The sensing system, in addition to the sensor body that can function as a biosensor electrode, may also include further elements, such as a reference electrode, a counter electrode, a voltage and/or current source, control elements and a means for reading or detecting changes in electrical conduction through the sensor body. The sensor body and/or other electrodes may in some instances be configured for various electrochemical observation techniques, including potentiometry, amperometry, and voltammetry, such as differential pulse voltammetry, cyclic voltammetry, alternating current voltammetry, and square wave voltammetry. In some aspects, the sensing system may further include controllers to provide currents and or voltages in working and/or reference electrodes within the proper operating parameters. Further components may include readout circuitry, data collection, and data storage components.

Preferably the sensor comprises a working electrode, a reference electrode and a counter electrode.

The reference electrode may be selected from: Ag/AgCl, Cu/CuSO₄, and Hg/HgSO₄. Preferably, the reference electrode is Ag/AgCl.

The counter electrode may be selected from: Pt (including Pt wires or meshes), and graphite (including graphite rods). Preferably, the counter electrode is Pt.

In some aspects, the sensor body is encased, sealed, covered, or partially covered, to provide a substantially leak-less or leak-free reference electrode. Preferably, the sensor body is encased, sealed, covered, or partially covered with glass, plastic, or a combination thereof, most preferably plastic. In some aspects, a substantially leak-less reference electrode may refer to an is encased, sealed, covered, or partially covered reference electrode that is stable over time, resistant to ethanol contact, or a combination thereof. A leak-less reference electrode that is stable over time may exhibit potential drift of less than about 100 mV, 80 mV, 70 mV, 60 mV, 50 mV, 40 mV, 30 mV, 20 mV, 10 mV over a period of at least about 30 days, 40 days, 50 days, or 60 days. A leak-less reference electrode that is resistant to ethanol exposure may exhibit potential drift of less than about 100 mV, 80 mV, 70 mV, 60 mV, 50 mV, 40 mV, 30 mV, 20 mV, 10 mV after contact with at least about 60%, 70% or 80% ethanol.

The sensor may be a micron, sub-micron, or nanoscale structure. Preferably, the sensor may be adapted to fit a well of a 24 well culture plate (less than about 16 mm diameter).

The sensor may be part of a flow system, wherein the solution or suspension is flowed over or through the sensor. The solution or suspension may be provided to the sensor in continuous or noncontinuous flow, preferably continuous flow.

In one embodiment, the sensor may comprise one working electrode. In another embodiment, the sensor may comprise a plurality of aptamer functionalised working electrodes, wherein each working electrode may comprise a different type of aptamer. In embodiments wherein the sensor comprises two or more working electrodes, wherein each working electrode comprises a different type of aptamer, the sensor may be adapted to detect two or more different types of target ligands.

In a particularly preferred embodiment, the sensor may comprise a leakless reference electrode, a counter electrode, and one or more working electrodes. The one or more working electrodes may include, but not be limited to: 1, 2, 3, 4, 5, 6, 7, 8, 9, 10 or more working electrodes. Each working electrode may comprise the same or different types of aptamers. Preferably, each working electrode comprises a different type of aptamer such that the sensor may be adapted to detect and/or measure 1, 2, 3, 4, 5, 6, 7, 8, 9, 10 or more different types of target ligands.

In some aspects, there is provided an array comprising a plurality of sensors. Each sensor may be the same or different. Preferably, the sensors are different. Preferably, each sensor comprises working electrode(s) with different types of aptamers. An array comprising 24 sensors, wherein each sensor comprises up to 3 working electrodes, and wherein each working electrode comprises a different type of aptamer, may be adapted to detect and/or measure up to 72 different types of target ligands.

The array may be disposed on a printed circuit board adapted for connection to a measuring or detecting device. Preferably, the board is adapted to fit a 24-well culture plate. Preferably, each sensor of the array is embedded in, or affixed to, the board and adapted to fit a well of the plate, thereby enabling reading of 24 wells of the plate with one measuring or detecting device.

All publications, including patents, published patent applications and non-patent literature referenced herein are incorporated by reference in their entirety.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Materials and Methods

Platinum wire (1 mm diameter) counter electrode, Ag/AgCl (3M NaCl) reference electrode, gold wire (0.20 mm diameter, length 0.5 m, high purity 99.99%) working electrode and 6-Mercapto-1-hexanol were purchased from Sigma-Aldrich. The aptamer sequence was purchased from Integrated DNA Technologies (Coralville, IA, USA). IDTE, EDTA, TCEP solution, Na₂HPO₄, NaCl, MgCl₂, NaOH, KCl, KH₂PO₄ and H₂SO₄ were purchased from commercial sources.

The potentiostat used was a CH 1000C and was purchased from CH Instruments (Austin, TX, USA).

### In Vitro Measurement for Characterisation of Phenylalanine

Signal measurements were conducted using Chl1040C 8 Channel Potentiostat Square-wave Voltammetry (SWV) mode and a standard three-electrode cell. SWV experiment was conducted with a potential step of 0.001, a potential window between-0.1 to −0.4 V, and an amplitude of 0.05 V with a frequency of 300 Hz. The experiment was performed in a working buffer (137 mM NaCl, 2.7 mM KCl, 10 mM Na₂HPO₄, and 1.8 mM KH₂PO₄ at pH 7.3).

### Example 1: Aptamer Sequence

For the electrochemical aptamer based phenylalanine sensor, an aptamer sequence for phenylalanine detection was used as shown below:

5′-CGACC-GCGTT-TCCCA-AGAAA-GCAAG-TATTG-
GTTGG-TCG-3′.

The 5′ end of the aptamer sequence was modified with a thiol-C6-SS group, which may form a thiol-Au bond to attach the aptamer to the surface of the gold electrode, and the 3′ end of the aptamer sequence was attached to methylene blue (a redox reporter) via a six-carbon linker as shown in FIG. 1.

### Example 2: Signal Measurement

The sensor used consists of a classic three electrode setup, a working electrode (WE), a counter electrode (CE), and a reference electrode (RE). The high Ag/AgCl electrode potential stability allows the potential of the other half-cell to be determined. A Pt electrode was used as the counter electrode, and was cleaned up with fire and then rinsed with Milli-Q water before use. A gold electrode was used as the working electrode. On the surface of the gold working electrode, a self-assembled monolayer (SAM) of an aptamer sequence was formed. The 5′ end thiol-C6-SS group of the aptamer sequence formed a thiol-Au bond with the gold electrode, thereby attaching the aptamer to the gold electrode. Binding of a target molecule (phenylalanine) to the aptamer (ie oligo probe) induces a structural/conformational change to the aptamer, which affects the electron transfer rate of the redox reporter attached to the 3′ end of the aptamer to the gold electrode, thereby changing the observed Faradic current. The structural change is shown schematically in FIG. 2, which is reflected by the aptamer sequence 3′ end redox reporter, methylene blue.

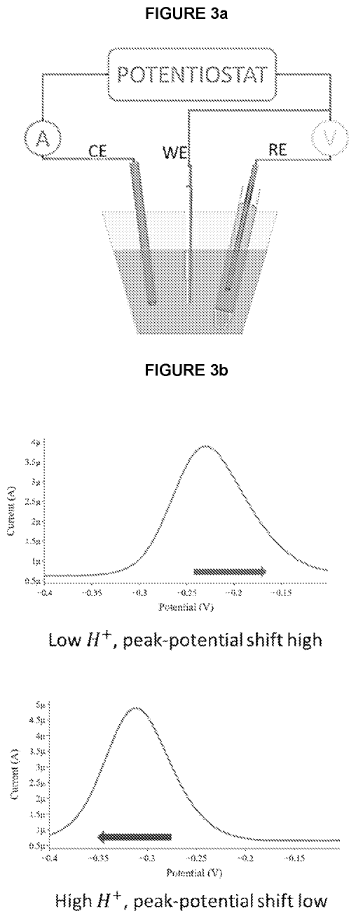

The variation of electron transfer efficiency may be measured by Faraday current. To detect the Faraday current, all three electrodes were connected to a potentiostat as shown in FIG. 3a. The potential of the working electrode (WE) was controlled versus the constant potential of the reference electrode (RE). The current flows between the working electrode and counter electrode (CE).

When the square wave voltammetry (SWV), which equals the square wave potential plus a staircase potential, was imposed on the WE, the Faradic current may be measured between the WE and CE. The corresponding peak potential shifts relative to the electrode mechanism. In an electrochemically reversible process, MB may be oxidized or reduced at different pH levels, especially via the 1e⁻/n H⁺ process at pH 6.0-10.7 (FIG. 3b). In such a way, the pH was reflected by the change of the peak-potential shift in the human physiologically relevant pH range (6.5-7.4).

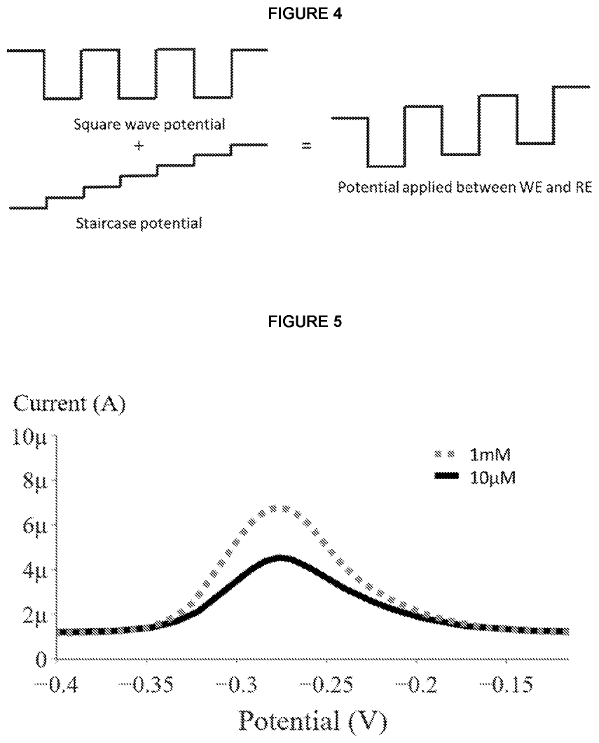

After connecting to the potentiostat, the Square Wave Voltammetry (SWV) measurement was performed on the device by the following method:
1. Shown in FIG. 4, the potentiostat applied a staircase potential plus a square wave potential between the working electrode and reference electrode.
2. The current was measured twice in one staircase between the counter electrode and the working electrode, once when pulsing forward and once when pulsing backward. The Faraday current was calculated by the difference between the forward current and the backward current. In theory, the magnitude of the peak current difference is proportional to the redox reporter methylene blue concentration:

$$Δ {i}_{p} = A ψ ( {k}^{∅} , φ , f , D )$$

$$where , A = {FSc}_{b , Red} \sqrt{D f}$$

where Δiₚ is the peak current difference, A is the amperometry constant, c_{b,Red} is the reduction species in the electrolyte solution bulk concentration, S is the electrode surface area, F is Faraday's constant, D is the diffusion coefficient which is related to the dimensional electrode kinetic, f is the frequency, k^{ø} is electrochemical rate constant, and ψ is a dimensionless mathematic parameter related to k^{ø}, φ, f, D.
3. Then, with a different concentration of target analyte, the structure of the SAM changed with an induced change of the differential peak current Δiₚ. The comparison between a high target analyte (phenylalanine) signal vs a low target analyte (phenylalanine) signal under a 300 Hz square wave frequency is shown in FIG. 5.

### Example 3: Electrochemical Aptamer Based Sensor Fabrication

The platinum electrode (1 mm diameter) was used as a counter electrode. The Ag/AgCl with NaCl filling solution was used as a reference electrode. The gold wire (200 μm diameter) was used as a working electrode.

Before binding the aptamer, the gold wire was cut to approximate 4-5 cm length and then the middle of the gold wire was covered by heat shrinking PTFE tube (McMaster-Carr Supply Company, Elmhurst, Illinois).

For coating the aptamer on the gold electrode, the following steps were used:
a) Resuspend the DNA oligo (modified aptamer sequence) in TE buffer (IDTE: 10 mM Tris, 0.1 mM EDTA, pH 8.0).
b) Reduce the thiol-modified oligo probe (100 μM, 2 μL) by treating the thiol-modified oligo probe for 1 hour in a solution of 2 μL, 10 mM TCEP solution at room temperature in the dark.
c) Dissolve the modified oligo in an “assembling buffer” (10 mM Na₂HPO₄ with 1 M NaCl and 1 mM MgCl₂ at pH 7.3) at a final concentration of 500 nM.
d) Electrochemical cleaning of the gold by the potentiostat: Pulse between-1V and −1.6V for 1500 segments with a scan rate of 1 V/s in 0.5 M NaOH solution to remove any organic residual on the gold electrode surface.
e) Increase the gold electrode surface roughness: 20 cycles between 0.35 and 1.6 V with a scan rate of 0.1 V/s, sample interval 0.0001s (no waiting between pulses) in 0.5 M H₂SO₄ to increase the electrode roughness.
f) Immerse the cleaned gold electrode in the 500 nM oligo probe for 1 hour in the dark.
g) Finally, in the dark, incubate the gold electrode in the assembling buffer (5 ml with 5 mM 6-mercaptoethanol overnight.

### Example 4: Measurements in Working Buffer

### Response Curve

Experimental titration curves were performed in the working buffer (137 mM NaCl, 2.7 mM KCl, ten mM Na₂HPO₄, and 1.8 mM KH₂PO₄ at pH 7.3). For each measurement, the sensors were scanned by potentiostat under SWV mode under two different frequencies 300 Hz (Initial E: −0.1V, Final E: −0.4V, Increments: 0.001V) and 10 Hz (Initial E: −0.1V, Final E: −0.4V, Increments: 0.003V).

After setting up the connections, measurements in the absence of phenylalanine (14.985 mL Working Buffer) were taken by interrogating the sensors more than 100 times until stable peak currents were obtained under both frequencies and this was treated as the stabilised Faraday peak current differences as

$$Δ {i}_{base}^{300} and Δ {i}_{base}^{10} .$$

After obtaining the base signal, the phenylalanine solution (Working Buffer base) was titrated from 1 nM to 3 mM as shown in Table 2. Between each concentration, one measurement was taken.

TABLE 2
Titration table in working buffer

| Desired | | 10 mM | | 1 mM | 100 uM | 10 uM | 1 uM | Buffer | | Total(ml) |
|---|---|---|---|---|---|---|---|---|---|---|
| 1 | nM | | | | | | 15 ul | 14.985 | ml | 15 |
| 3 | nM | | | | | | 33 ul | 967 | ul | 16 |
| 10 | nM | | | | | | 122 ul | 878 | ul | 17 |
| 30 | nM | | | | | 37 ul | | 943 | ul | 18 |
| 100 | nM | | | | | 136 ul | | 864 | ul | 19 |
| 300 | nM | | | | 41 ul | | | 959 | ul | 20 |
| 1 | uM | | | | 150 ul | | | 850 | ul | 21 |
| 3 | uM | | | 45 ul | | | | 955 | ul | 22 |
| 10 | uM | | | 160 ul | | | | 836 | ul | 23 |
| 30 | uM | 49 | ul | | | | | 951 | ul | 24 |
| 100 | uM | 178 | ul | | | | | 822 | ul | 25 |
| 300 | uM | 530 | ul | | | | | 470 | ul | 26 |
| 1 | mM | 2.22 | ml | | | | | 780 | ul | 30 |
| 3 | mM | 9 | ml | | | | | 1 | ml | 40 |

After finalizing the measurements, the raw signal of each concentration was converted into a Normalized current change differential ratio Dᵢ:

$${D}_{i} = ( \frac{Δ {i}_{phe}^{300}}{Δ {i}_{base}^{300}} - \frac{Δ {i}_{phe}^{10}}{Δ {i}_{base}^{10}} ) / 0.5 ( \frac{Δ {i}_{phe}^{300}}{Δ {i}_{base}^{300}} - \frac{Δ {i}_{phe}^{10}}{Δ {i}_{base}^{10}} )$$

The Dᵢ correlates with the Langmuir-Hill Equation, which relates to the titration result:

$${D}_{i} = {D}_{imin} + \frac{( {D}_{imax} - {D}_{imin} ) * {PHE}^{n H}}{{PHE}^{n H} + {D}_{\frac{1}{2}}^{n H}}$$

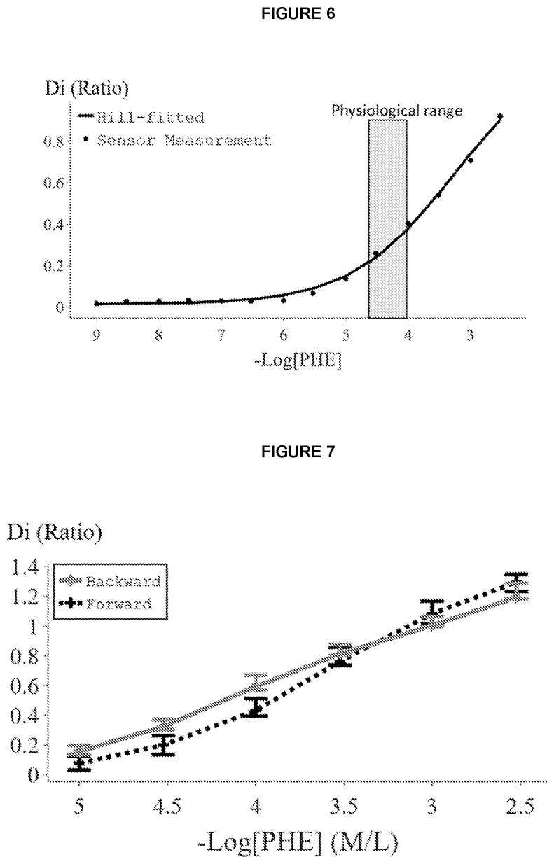

FIG. 6 shows the titration measurement results for the aptamer sensor, where the dot is the measured point, and the solid line is the Langmuir-Hill fitted curve. From the titration curve, the sensor shows its potential to measure a physiological phenylalanine concentration range from 30 μM to 300 μM.

### Sensor Key Characteristics

The sensor hysteresis effect was examined and results are shown in FIG. 7. After gold electrode fabrication and incubation overnight, the sensor was measured under SWV from high phenylalanine concentration to low phenylalanine concentration (Forward, 3 mM-10 μM) and low phenylalanine concentration to high phenylalanine concentration (Backward, 10 μM-3 mM). For each concentration point, 20 measurements were taken. The results show negligible hysteresis on phenylalanine concentration variation for this sensor to detect phenylalanine.

### Sensor Repeatability

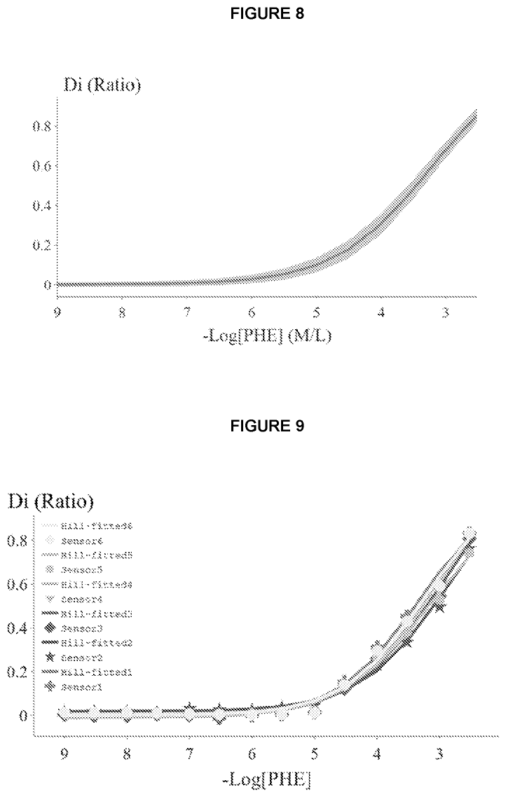

The repeatability of the sensor was tested with 6 gold electrodes (Sensor 1-6) with aptamer recoating performed 3 times (fabrication and incubation overnight) and the titration process as described by the present disclosure on the 6 gold electrodes simultaneously. FIG. 8 shows the repeatability of the electrode with respect to aptamer recoating. FIG. 9 shows the repeatability with respect to different electrodes. The results indicate that under a standard fabrication process, the aptamer sensor can reach a stable response curve, so calibration is not required after each fabrication.

### Example 5: Aptamer Based Dual pH Sensor

pH is a critical physiological factor that is affected by both cell metabolic and drug activity, which is prevalent and integrated into Lab-on-a-chip (LoC) for drug screening.

### In Vitro Measurement for Characterization of pH

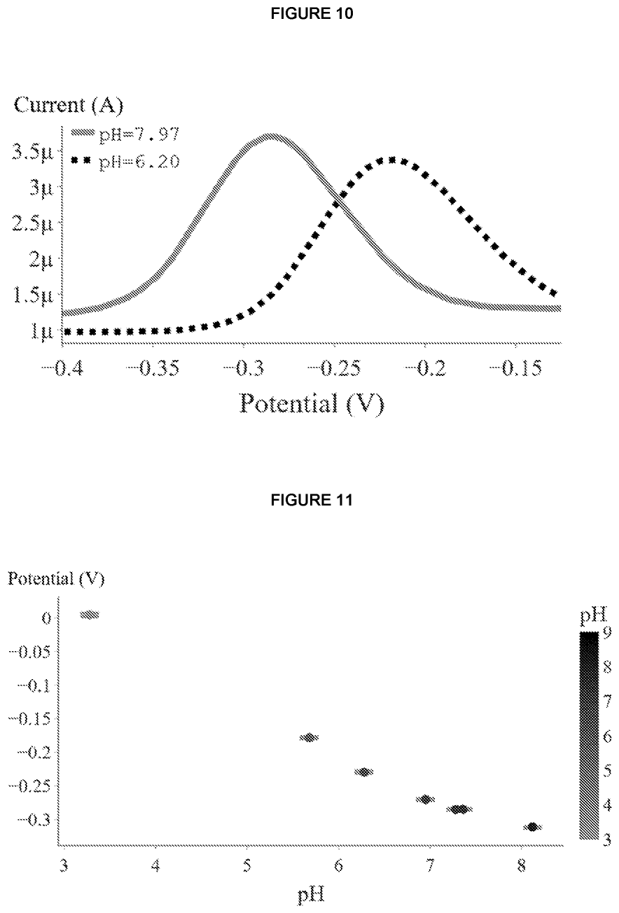

Signal measurements were conducted using Chl1040C 8 Channel Potentiostat SWV mode and a standard three-electrode cell as described in Example 4. The pH was reflected by the variation of the peak potential as shown in FIG. 10. SWV was performed in a “Working Buffer” with different pH values (7.97 and 6.20). The number of protons involved in the Methylene Blue reversible redox reprocess is pH dependent. With a higher pH value (pH=7.97 in the plot), the peak potential increased, with a lower pH value (pH=6.20), the peak potential decreased.

### Titration and Calibration Curve for pH Measurement

To further confirm the pH detection range, after gold electrode fabrication and incubation, the response curve was obtained by adding 0.5 ml of hydrogen chloride or sodium hydroxide to adjust the pH level in 20 ml of “Working Buffer” with pH=7.4. After each addition, the reference pH was measured using a commercial pH meter, and each dot point in the plot was the mean of 20 successive measurements performed in the same pH solution. The results are shown in FIG. 11.

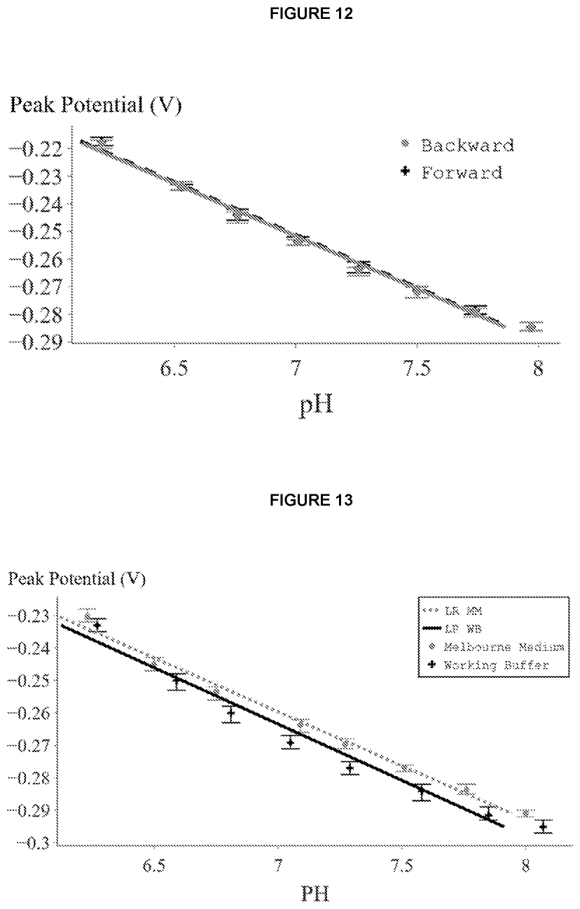

The pH calibration curve was measured in a “Working Buffer” with varying pH values from 6 to 8. The solution was sterilized and prepared in advance. In FIG. 12 forward was measured from low pH to high pH, and backward was measured from high pH to low pH value. Before moving between different pH solutions, the sensor was rinsed with DI water. The LR Backward and LR Forward are the line fitted with logistic regression respectively to the Backward data and the Forward data. Each solution was measured with 20 groups of data under SWV 300 HZ.

The results in FIG. 12 show the sensor peak potential measurement has excellent linear relationship with pH and negligible hysteresis over pH variation.

### Functional Capability in Different Medium

FIG. 13 shows the assessment of functional compatibility in different types of cell culture medium with the same sensor. After fabrication of the gold electrodes, the pH calibration curve measured in “Working Buffer” and “Melbourne Medium” were conducted with varying pH values from 6 to 8. The solution was sterilized and prepared in advance with different pH values. Before moving between different pH solutions, the sensor was rinsed with DI water. The LR MM and LR WB are the lines fitted with logistic regression respectively to the “Working Buffer” data and the “Melbourne Medium” data. Each solution was measured with 100 groups of data under SWV 300 HZ.

The results confirm the pH detection ability in both complex Melbourne Medium and Working Buffer. As the response curves are similar in the Working Buffer and the Melbourne Medium, this suggests the pH sensor may work in a complex cell culture.

### Evaluation of Sensor Stability

To evaluate the sensor's stability, after the sensor's overnight fabrication, 100 successive SWV were measured within a 10-minute period in 5 different pH values which were pre-adjusted in “Working Buffer” with values pH=6.27, pH=6.59, pH=7.05, pH=7.58, pH=8.07, and 100 successive SWV were measured within a 10-minute period in 5 different pH values which were pre-adjusted in “Melbourne Medium” with value pH=6.23, pH=6.50, pH=7.09, pH=7.51, pH=8.00. The measurement potential was relatively stable for each pH solution.

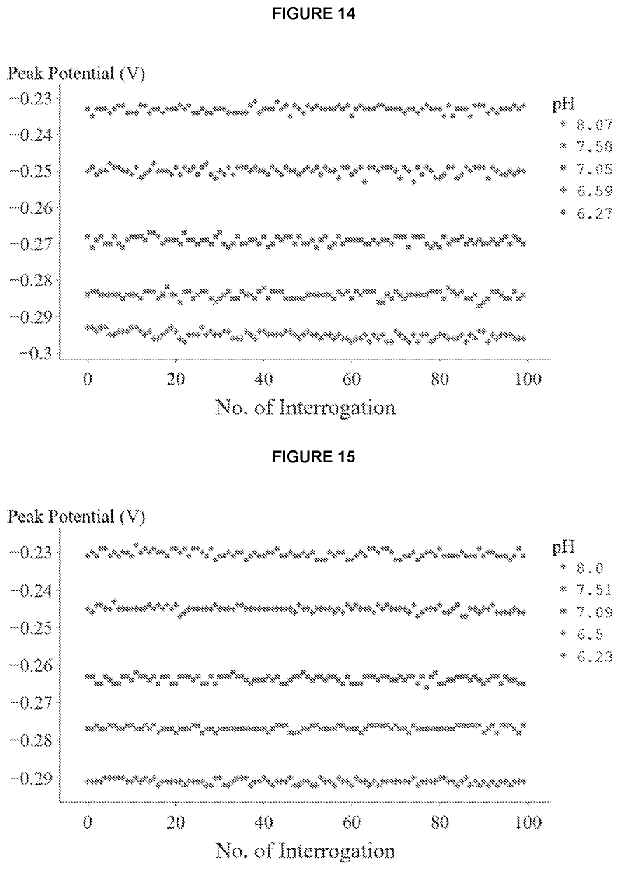

FIG. 14 shows the sensor reproducibility for an individual sensor measuring the pH in the “Working Buffer”. 100 successive SWV were measured within 10 minute periods in 5 different pH values which were pre-adjusted in the “Working Buffer” with pH values of pH=6.27, pH=6.59, pH=7.05, pH=7.58, pH=8.07 (from bottom to top).

FIG. 15 shows the sensor reproducibility for an individual sensor measuring pH in the “Melbourne Medium”. 100 successive SWV were measured within 10 minute periods in 5 different pH values which were pre-adjusted in the “Melbourne Medium” with pH values of pH=6.23, pH=6.50, pH=7.09, pH=7.51, pH=8.00 (from bottom to top).

Evaluation of the Response Curve with Different Aptamer Sequences

To show that the device was not specific to the phenylalanine aptamer sequence, two more experiments were conducted to show that other aptamer sequences modified by methylene blue are responsive to pH. Two electrodes coated with separate aptamer sequences-Doxorubicin aptamer and Tryptophan aptamer-were fabricated according to the fabrication process described in Example 3.

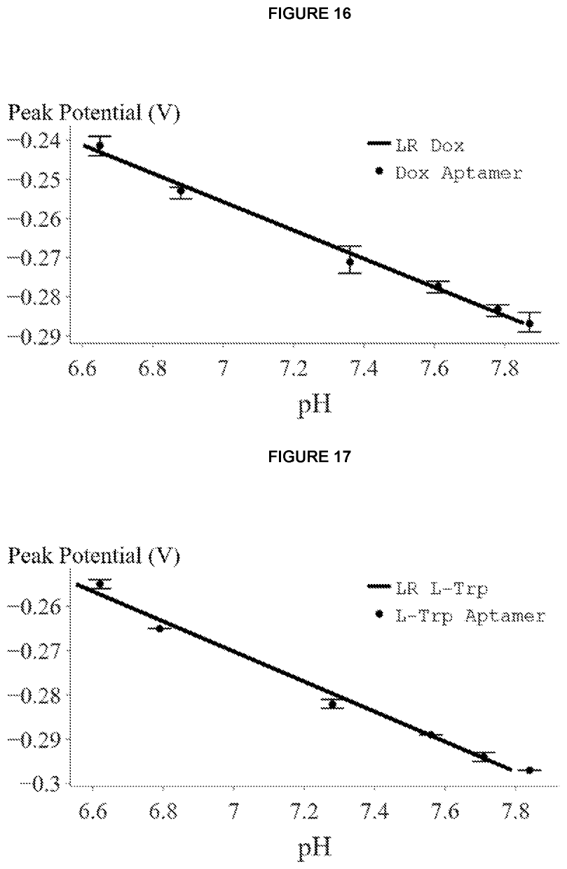

FIG. 16 shows the results of the potential at various pH measurements for the Doxorubicin aptamer probe. This was conducted by using six “Working Buffer” solutions with different pH values (pH=7.87,7.78, 7.61, 7.36, 6.88, and 6.65). Each solution was measured with 10 groups of data under SWV with a 300 Hz frequency.

FIG. 17 shows the results of the potential at various pH measurements for the L-tryptophan aptamer probe. This was conducted by using six “Working Buffer” solutions with different pH values (pH=7.84,7.71, 7.56, 7.28, 6.79, and 6.62). Each solution was measured with 10 groups of data under SWV with a 300 Hz frequency.

### Example 6: On-Chip Devices

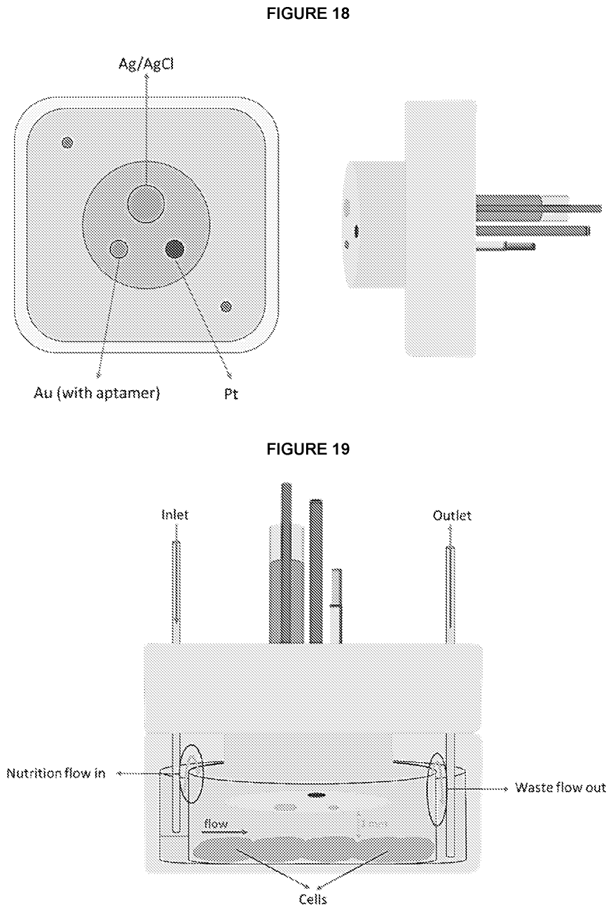

On-chip sensor devices may be made to directly measure cell culture pH and phenylalanine levels in real-time as shown schematically in FIG. 18 and FIG. 19.

FIG. 18 shows a bottom view (left) and a side view (right) of an on-chip sensor well lid. The cylinder in the middle is the supporter and holder for Ag/AgCl electrode, Au electrode, and Pt electrode to insert in and generate an approximate plane surface. There are two holes located on opposite sides of the cylinder to insert a tube for nutrition and waste flow.

FIG. 19 shows an on-chip sensor well overview. There are three flutes in the perfusion well plate. One flute is for the nutrition inlet, when the flute liquid level is greater than the second middle-round flutes, the medium will flow in the cell culture flute gradually. When the second middle-round flutes medium exceeds its flute wall, the waste medium will flow out and be ejected into the waste reservoir. The sensor-well lid covers the perfusion well plate with a 1 mm distance between the cells on the bottom well, to precisely measure the cell environment bio information as well as protect the SAM layer on the gold electrode surface. The flat sensor lid surface and the small distance between the cell and sensor allows the laminar flow over the cell surface.

On-chip devices can be used for the real-time monitoring of the cell environment and to detect cell pH and phenylalanine concentration in the flowing medium supply. An on-chip sensor may be used to measure different target molecules by exchanging aptamer probes in a modular manner. Moreover, the sensor housing assembly may be modified to accommodate up to 8 sensors affording measurement of multiple analyte targets in real-time.

### Example 7: Sensitivity Prediction

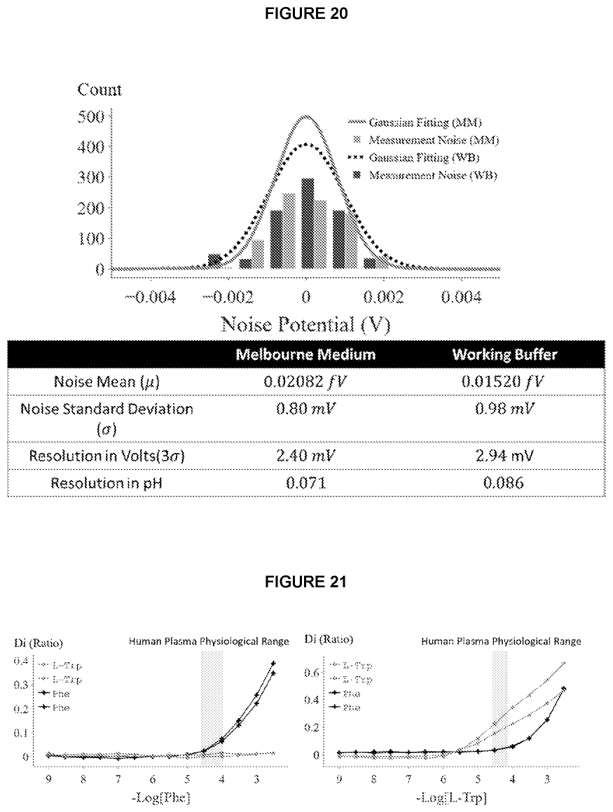

1000 measurements in Melbourne Medium and Working Buffer under different pH condition were taken. The mean potential was measured for each pH and used to extract each pH condition's noisy potential by subtracting the measurement potential from the mean potential. The noisy potential signals were fitted into Gaussian Noise distribution model. From the fitted model, the standard deviation (σ) and mean (μ) of the noise potential was calculated. Based on the standard deviation (σ) and sensor slope (0.037 mV/pH), the resolution was predicted, which is 0.08 pH (three-sigma law, 99.7% prediction accuracy) (FIG. 20).

### Example 8: Measurement of at Least 2 Analytes at the Same Time with a Dual Biosensor

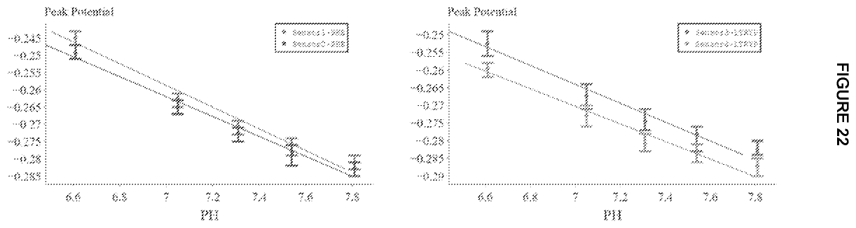

To confirm the sensor can measure at least 2 analytes at same time, two experiments were conducted. In experiment 1, two electrodes covered with phenylalanine aptamer and another two electrodes covered with L-tryptophan aptamer were fabricated and incubated overnight. All four electrodes were tested in the same conditions at the same time. In the titration process, phenylalanine (from 1 nM to 3 mM) was titrated first in “Working Buffer” and then the L-tryptophan (from 1 nM to 3 mM) was titrated in “Working Buffer”. This result indicates that between the phenylalanine aptamer sensor and the L-tryptophan aptamer sensor there are negligible influences, especially in the target measurement range (FIG. 21).

After the titration process in experiment 1, part 1, sensors were taken into working buffer under different pH values (6.61, 7.05, 7.31, 7.54, 7.81), at each pH, 10 measurements were taken. From the results, both PHE and L-TRP aptamer show the measurement of pH after PHE and L-TRP titration (FIG. 22).

In experiment 2, two electrodes covered with phenylalanine aptamer and another two electrodes covered with L-tryptophan aptamer were fabricated and incubated overnight. All four electrodes were tested in the same conditions at the same time. In the titration process, the L-tryptophan (from 1 nM to 3 mM) in “Working Buffer” was first titrated, then phenylalanine (from 1 nM to 3 mM) in “Working Buffer” was titrated second.

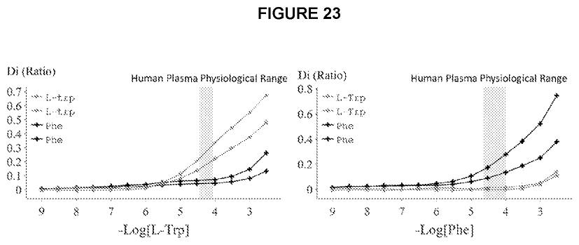

This result confirmed that between the phenylalanine aptamer sensor and L-tryptophan aptamer sensor there was negligible influences in the interested measurement range (FIG. 23).

After the titration process in experiment 2, sensors were taken into working buffer under different pH values (6.81, 6.99, 7.21, 7.38, 7.54), at each pH, 10 measurements were taken. From the results, both the PHE and L-TRP aptamer all show the measuring of pH after PHE and L-TRYP titration (FIG. 24).

### Example 9: Dynamic Condition Testing

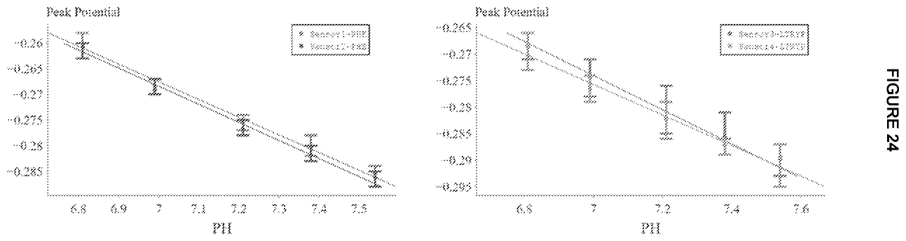

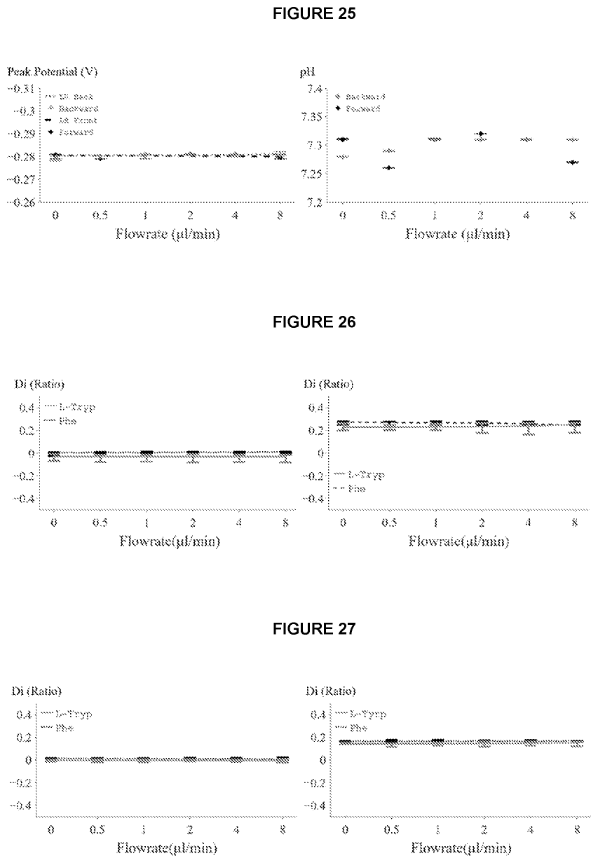

To integrate the sensor into a dynamic in vitro system, the inventors confirmed whether the flowrate has an influence on the measurement. An experiment was designed which allows the sensor in the on-chip devices to take 10 groups of measurement in pH=7.3 working buffer under 6 different flowrates: 0 μl/min, 0.5 μl/min, 1 μl/min, 2 μl/min, 4 μl/min, 8 μl/min. The flowrate was achieved and controlled by a microfluidic pump and varied from low to high and high to low accordingly. From the results in FIG. 25, the peak potential was relatively stable in an acceptable range from −0.281V to −0.279V, and its converted pH range from 7.26 to 7.32, which proves the sensors ability to retain measurement stability with flowrate variation.

For target analyte stability under dynamic conditions, PHE and L-TRP aptamer coated electrodes were placed in a 24-cell well, starting with static conditions and then increased to 0.5 μl/min, 1 ulmin, 2 μl/min, 4 μl/min, and 8 μl/min. In this experiment, two concentrations of target analytes were tested (0 μM and 100 μM). Under each concentration's flowrate, 5 measurements were taken. From the results in FIG. 26, the “Forward” (flowrate change from low to high) dynamic condition has negligible influence on the sensor's measurement ability.

In another experiment, the flowrate is conducted “Backward”, so the flowrate starts with 8 μl/min, decreased to 4 μl/min, 2 ulmin, 1 μl/min, 0.5 μl/min, and then static conditions. In this experiment, two concentrations of target analytes were tested (0 μM and 100 μM). Under each concentration's flowrate, 5 measurements were taken. From the results in FIG. 27, the “Backward” (flowrate change from high to low) dynamic condition also has negligible influence on the sensor's measurement ability.

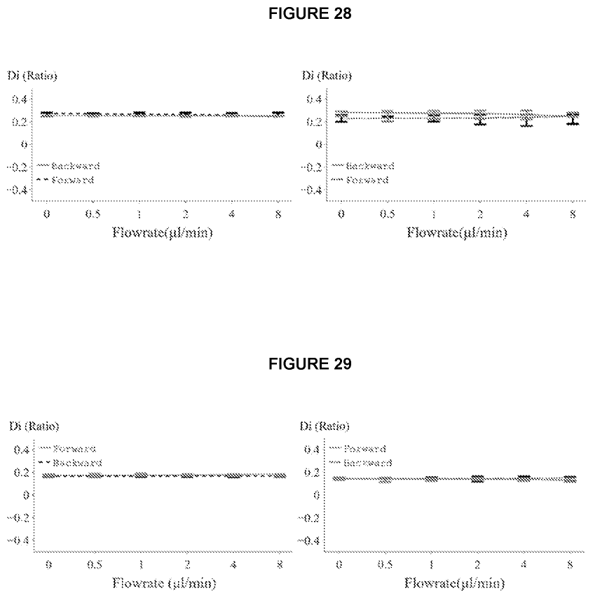

For the flowrate hysteresis test on the target analytes the PHE aptamer electrode and L-TRP aptamer electrode ware placed into 100 μM “Working Buffer”. The flowrate was changed from “Forward” (flowrate change from low to high) to “Backward” (flowrate change from high to low). Under each flower rate, 5 measurements were taken. From the results, the flowrate doesn't generate any appreciable hysteresis effect on the aptamer sensor's measurement for flowrate changes from “Forward” to “Backward” (FIG. 28).

In a further experiment, the PHE aptamer electrode and L-TRP aptamer electrode ware placed into 100 μM “Working Buffer”. The flowrate was changed from “Backward” (flowrate change from high to low) to “Forward” (flowrate change from low to high). Under each flowrate, 5 measurements were taken. From the results, the flowrate doesn't generate any appreciable hysteresis effect on the aptamer sensor measurement from “Backward” to “Forward” (FIG. 29).

### Example 10: Cell Metabolism Measurement

To ascertain the measurement ability and biocompatibility of the dual-pH sensor in vitro, cell metabolism measurements were conducted.

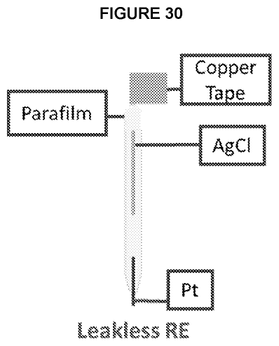

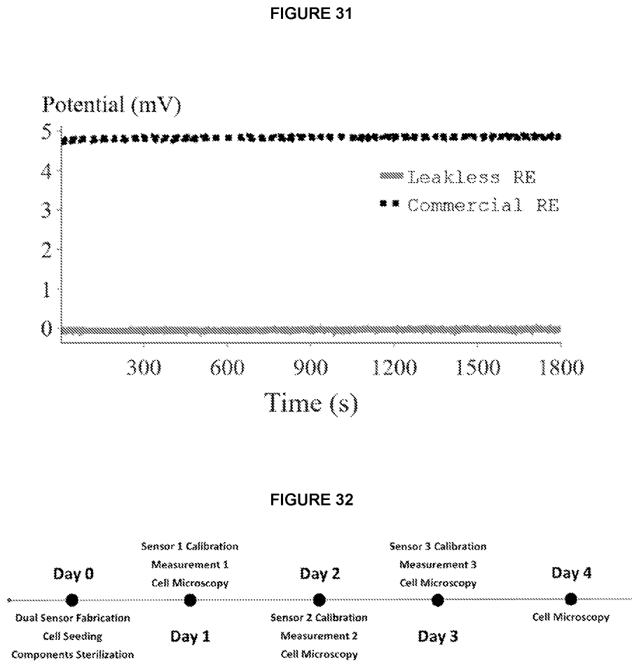

Before the experiment, a leak-less reference electrode was fabricated. A torch was used to seal one end of a capillary glass tube with platinum wire. Then the opposite end was filled with 3M NaCl and an electrochemical coated Ag/AgCl wire was inserted into the glass tube. The end with the Ag/AgCl wire was sealed by parafilm, and the exposed Ag wire was covered with copper tape. As shown pictorially in FIG. 30. To confirm the leak-less reference electrode stability, the open circuit potential was measured in 3M NaCl against a commercial reference electrode before being used in cell culture measurements (FIG. 31).

The overview of the measurement timeline is shown in FIG. 32. The cell was first seeded on day 0 to ensure the full confluency before the measurement date. Meanwhile, on day 0, a gold electrode coated with the phenylalanine aptamer was fabricated and incubated overnight. All the fabrication kits were sterilized and filtered before the fabrication and all the other components were sterilized by ethanol and UV for 1 hour before being put into the measurement incubator (5% CO₂, 37 Celsius). On Day 1 the cell medium was changed to Melbourne Medium and the cell status confirmed by microscope before being put into an incubator and the first calibration for electrode 1. For each calibration, the sensor was calibrated in the incubator under 2 different pH buffered solutions and 2 concentrations of phenylalanine with 0 μM, 10 μM and 100 μM respectively in Melbourne Medium. The calibration process and microscopy of the cell was repeated for electrode 2 and 3 on day 2 and day 3. Between each day, 5 hours of continuous measurement was performed at 15 minute intervals. The experiment stopped on day 4 and the cell microscopy was taken to confirm the cell health status.

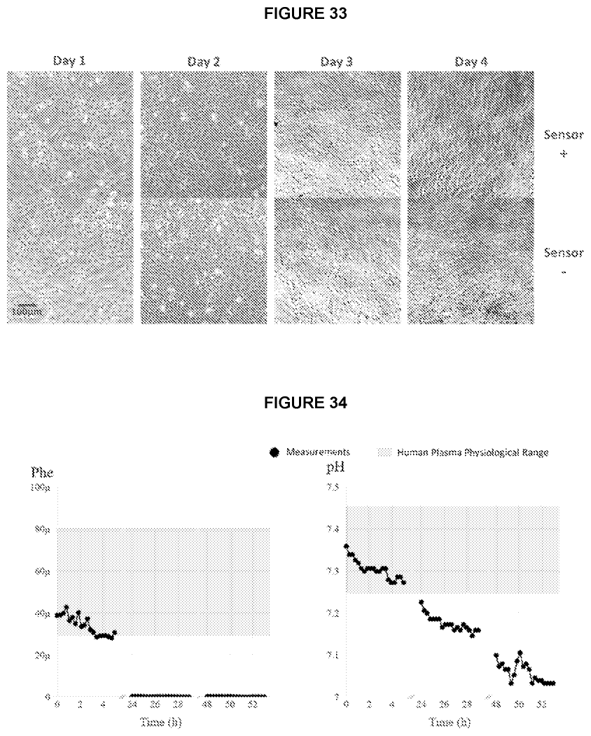

From a comparison of the cell microscopy images with the sensor well and without the sensor well (FIG. 33) there was no significant difference in morphology or viability. These data confirmed the sensor's biocompatibility and non-cytotoxicity.

The measurement of phenylalanine (FIG. 34, left), on the first day for the first 5 hours shows the cell culture closely stays in physiological range to maintain cell growth and cell division. On day 2, the phenylalanine level reduces to 1 μM in hypo physiological range, as expected, as cell growth and division requires amino acid consumption.

From the measurement of the pH (FIG. 34, right), as cells consume the nutrients from the culture medium, metabolic by-products and waste are generated, including organic acids such as lactic acid. If the rate of nutrient consumption exceeds the supply of fresh medium, the accumulation of metabolic by-products can lower the pH.

The sensor's ability to reflect the cell metabolism has been shown and confirms that regular monitoring of nutrient levels, pH, and waste accumulation is important to ensure optimal cell growth and maintain a healthy cell culture in long time experiments to improve prediction efficacy.

### Example 11: Continuous Flow Sensor

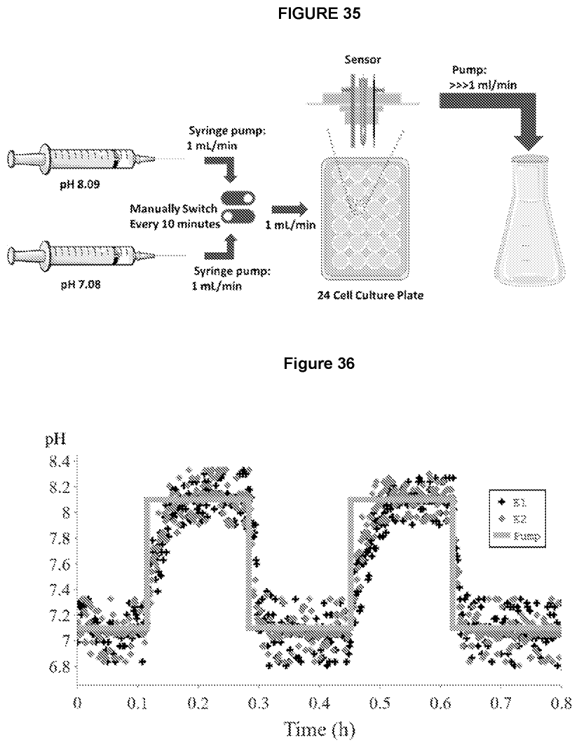

To develop an example of a real-time, continuous flow measurement of a dual-purpose sensor, two syringe pumps containing solutions of different pH were loaded (FIG. 35) and connected to a manually controlled valve. The flowrate of each syringe pump was 1 mL/min, and the pump solution was manually switched every 10 minutes. The output of the valve was connected to the input of one well of the 24-well culture plate. The output of the well was aspirated by a line that had higher flow rate than the input flow rate. To ensure the sufficiency of the medium volume inside the well in covering the sensor, the input tubing was at a lower height than the output tubing and was higher than the sensor level.

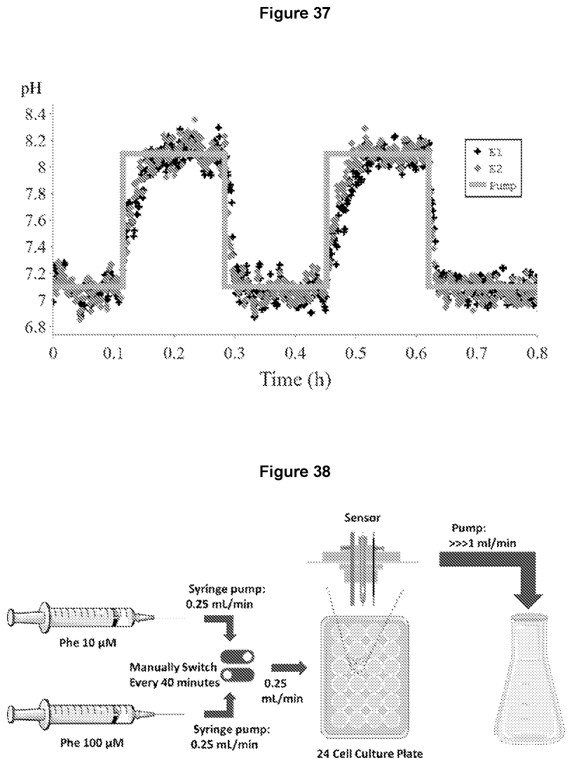

Results were obtained from two sensor electrodes (from within the same well) (black and grey); the grey curve is the theoretical pH (FIG. 36). The pH measurement afforded by the two separate electrodes closely matches the theoretical prediction. However, the signal showed some fluctuation possibly due to the high flowrate generating perturbations on the sensor surface. After Savitzky-Golay filtering, the pH estimations were more stable (FIG. 37).

The phenylalanine real-time measurement was achieved in a similar setting to that used for real-time pH measurements. The syringe-pumps contained 10 μM phenylalanine or 100 μM phenylalanine in working buffer. The flowrate was 0.25 mL/min (as the scan rate for phenylalanine measurement is slower than pH measurement), and the syringe pump perfusing the well containing the sensor was manually switch every 40 minutes (FIG. 38).

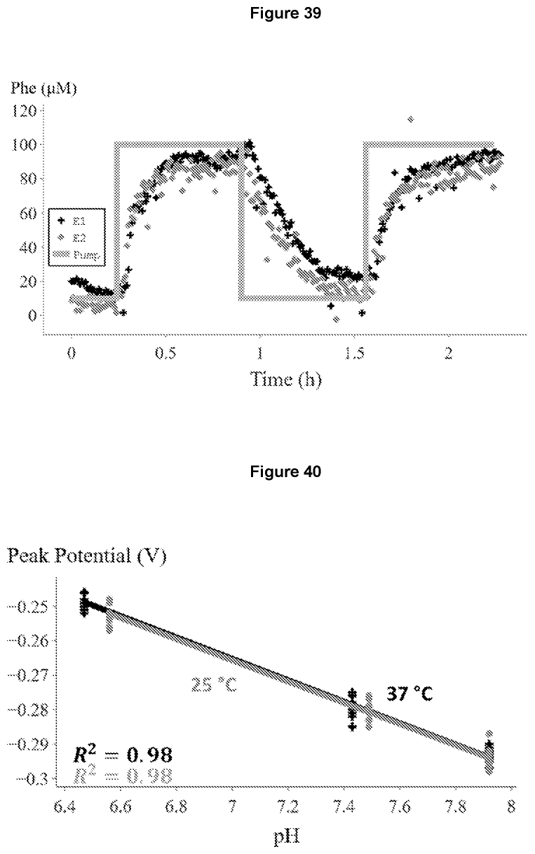

The phenylalanine real-time measurement of the two separate electrodes (black and grey), closely matched the predicted value (grey) line (FIG. 39).

### Example 12: PH Sensing Temperature Dependence Experiment

The sensor was equilibrated in the incubator (5% CO2, 37° C.), in Melbourne Medium (human plasma like medium) before being exposed to a step wise increase in pH. The pH response curve in the incubator in Melbourne Medium was first tested. Both working buffer and Melbourne media with 3 different pH values (working buffer pH 6.48, pH 7.42, pH 7.91) were prepared and with 3 different pH values (Melbourne Medium: pH 6.55, pH 7.50, pH 7.91). The same sensor was first tested in the working buffer under ambient temperature and then tested in Melbourne Medium in incubator. For each pH value, 10 SWV scans under 300 Hz were taken. The two fitted curves showed no significant differences (FIG. 40).

### Example 13: Sensor Phenylalanine Response Curve in Human Plasma Like Conditions

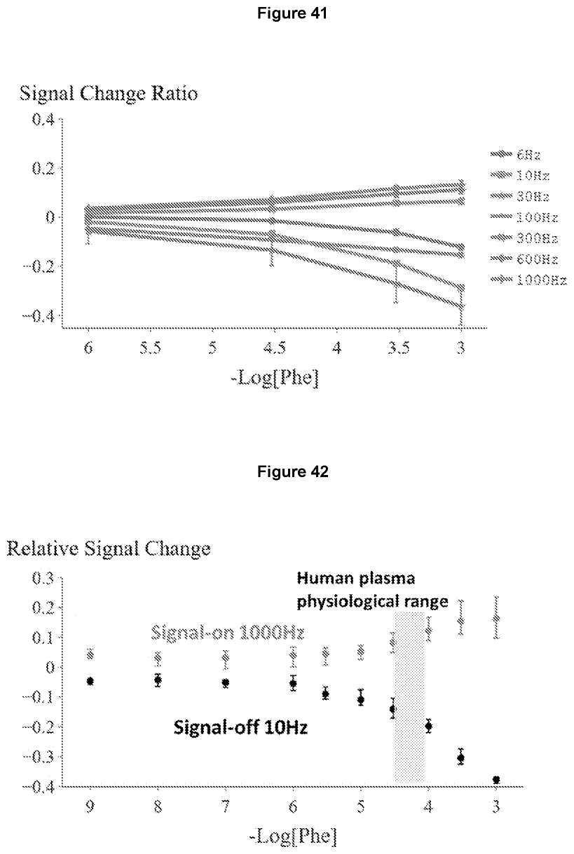

To find the optimal response frequency for the sensor to measure phenylalanine in human plasma like conditions, the sensor was scanned five times under different frequencies (6 Hz to 1000 Hz) in phenylalanine concentrations from 1 μM to 1 mM in Melbourne Medium. The 1000 Hz and 10 Hz were deemed to be the optimal signal-on and signal-off frequencies, respectively, under human plasma like conditions (FIG. 41).

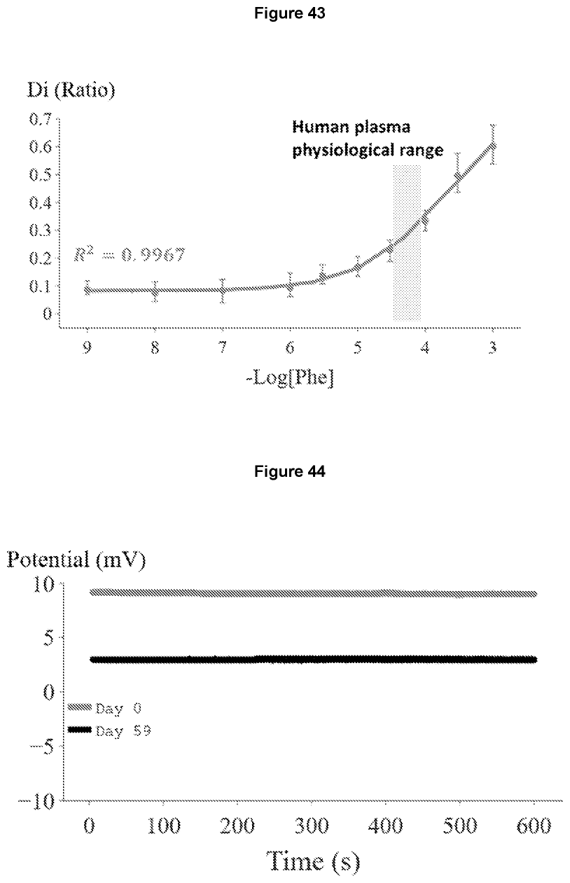

After obtaining the optimal frequencies, the response curve for phenylalanine was obtained by preparing different stock solutions of phenylalanine in Melbourne Medium with phenylalanine concentrations of: 10 mM, 1 mM, 300 μM, 100 μM, 30 μM, 10 μM, 1 μM, 100 nM, 10 nM. For the titration curve, a pre-set pod with sterilized 14 ml of Melbourne Medium in incubator (37° C., 5% CO₂), was equilibrated for 1 hour for the medium in the incubator environment. Measurements were then obtained with an apparatus comprising a three-electrode structure in the pod connected to the potentiostat, and a long silicon tube was connected between the pod and a reservoir of Melbourne Medium in a syringe in the incubator fitted with a filter to maintain sterility. The syringe titration (Table 3) was then performed. The sensor phenylalanine detection with 1000 Hz turn-on frequency performed with higher stability and sensitivity compared to turn-on frequency 300 Hz. (FIG. 42 and FIG. 43).

TABLE 3
Table of titration for phenylalanine concentrations
change from 1 nM to 1 mM in Melbourne Medium.

| Stock | | Stock Adding | MM | | MM | Desired | | Total |
|---|---|---|---|---|---|---|---|---|
| Concentration | | Volume | Concentration | | Volume | Concentration | | Volume |
| 10 | nM | 1.56 ml | 0 | nM | 14.0 ml | 1 | nM | 15.6 ml |
| 100 | nM | 1.73 ml | 1 | nM | 15.6 ml | 10 | nM | 17.3 ml |
| 1 | μM | 1.92 ml | 10 | nM | 17.3 ml | 100 | nM | 19.2 ml |
| 10 | μM | 2.13 ml | 100 | nM | 19.2 ml | 1 | μM | 21.3 ml |
| 30 | μM | 2.37 ml | 1 | μM | 21.3 ml | 3 | μM | 23.7 ml |
| 100 | μM | 2.63 ml | 3 | μM | 23.7 ml | 10 | μM | 26.3 ml |
| 300 | μM | 2.93 ml | 10 | μM | 26.3 ml | 30 | μM | 29.3 ml |
| 1 | mM | 3.25 ml | 30 | μM | 29.3 ml | 100 | μM | 32.5 ml |
| 3 | mM | 3.60 ml | 100 | μM | 32.5 ml | 300 | μM | 36.1 ml |
| 10 | mM | 3.98 ml | 300 | μM | 36.1 ml | 1 | mM | 40.1 ml |

### Example 14: Leakless Reference Electrode Characteristics

The long-term stability of a glass leakless reference electrode was tested. After the first day of fabrication, the leakless reference electrode potential was compared with a commercial reference electrode in 3M NaCl. Then the leakless reference electrode was stored under ambient environmental conditions. After 2 months, another potential measurement was taken. The leakless reference electrode was observed to have less than 9 mV potential drift (FIG. 44).

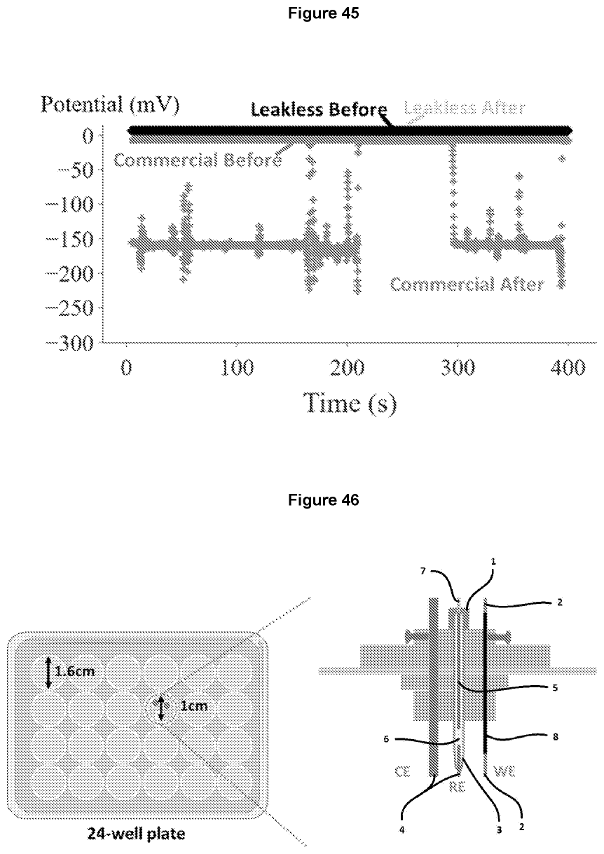

Ethanol is frequently employed for the sterilization of biosafety lab equipment. The stability of the leakless reference electrode exposed to ethanol was tested. The open circuit potential (OCP) measurement of leakless reference electrode and commercial reference electrode was first taken before the reference electrodes were sprayed with 80% ethanol. Then the leakless reference electrode and commercial reference electrode were sprayed with 80% ethanol. Another OCP measurement of leakless reference electrode and commercial reference electrode was taken to compare the influence of ethanol spray. The leakless electrode potential appears to be unaffected by ethanol spray, whereas the commercial reference potential became unstable (FIG. 45). These observations confirmed that the leakless reference electrode has a greater hermetic seal.

### Example 15: Design of Sensor Holder for Measurement in Cell Culture

The sensor detection element was miniaturized to fit into a single well (15.6 mm diameter) of a 24-cell-well plate (CoStar, Washington, USA) (FIG. 46). The sensor detection element comprises three electrodes: counter electrode (Pt), reference electrode (Ag/AgCl), and working electrode (Au). The 3D printed sensor holder fixes the position of counter electrode and working electrode by screw; the reference electrode was sealed and fixed by parafilm. The stable arrangement of the three electrodes guarantees that the sensor remains in a fixed position, preventing direct contact between the sensor and the cell (FIG. 46). This configuration minimizes the potential for interference during system operation, such as sensor scratches affecting cell growth.

### Example 16: Real-Time Cell Culture Measurement

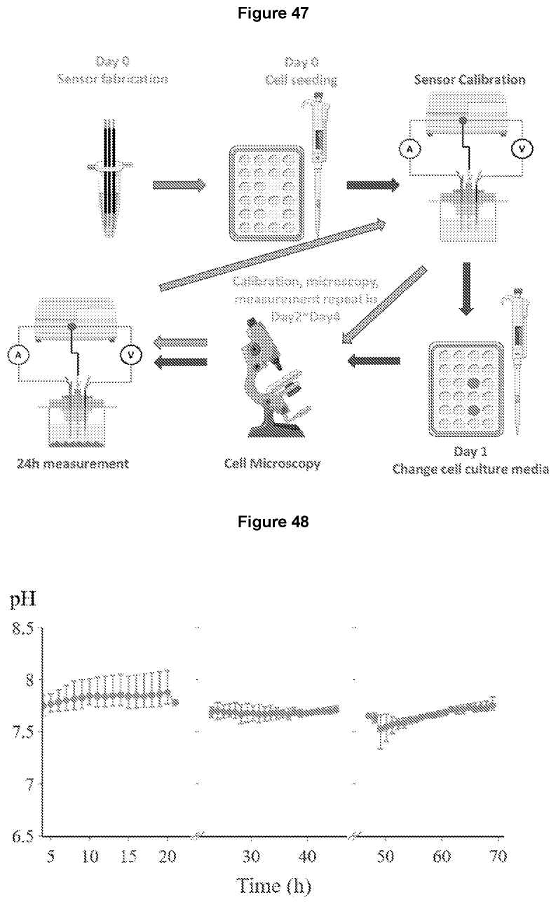

The timeline and methods are shown in FIG. 47. Melbourne Medium phenylalanine calibration was conducted to generate a curve with 0.10 and 100 μM. Working Buffer with pH 6.5 & 7.5 was prepared for pH calibration. A549 cells (human adenocarcinoma alveolar basal epithelial cells) were seeded on 24 well plates at 10000 cell/well seeding density. On day 1, Melbourne Medium was pre-equilibrated for 2 hours. Then the first group of sensors were calibrated in Working Buffer with scan frequency 1000 Hz. Then sensors phenylalanine sensing was also calibrated (0 μM, 10 μM, 100 μM) in Melbourne Medium with signal-on frequency 1000 Hz and signal-off frequency 10 Hz. The A549 cell culture medium was changed to 1.5 ml Melbourne Medium and cells were observed and imaged under brightfield light microscopy. Real-time in situ measurement in cell culture was initiated at hourly intervals. The electrodes were freshly calibrated each day, with daily cell imaging. This whole calibration-measurement process was repeated for 3 days.

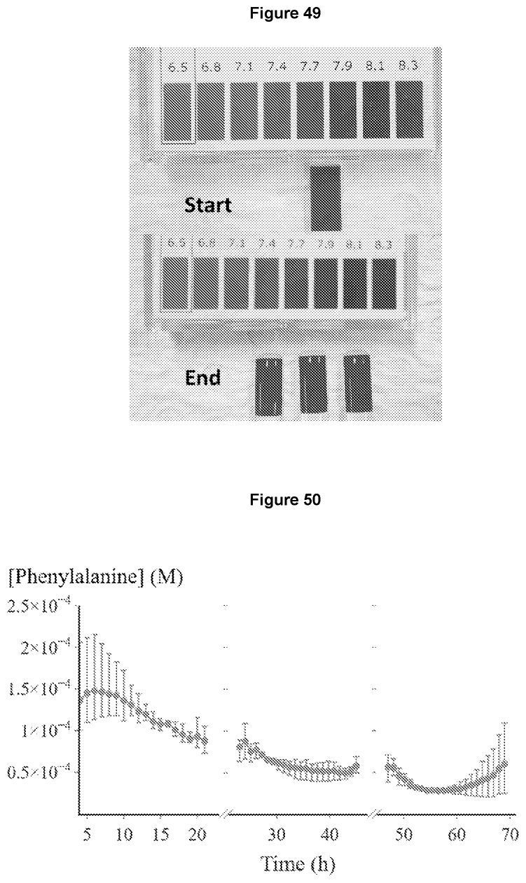

The pH of A549 cell culture remained relatively stable over 72 hours (FIG. 48, n=3), as benchmarked by pH strip (FIG. 49).

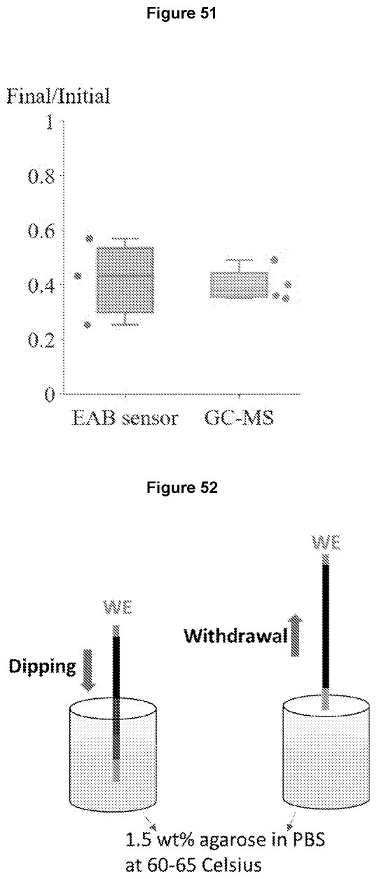

The sensor results as shown in the phenylalanine measurement reflect declining phenylalanine levels, consistent with the consumption of the cell medium for cellular metabolism (FIG. 50, n=3). The initial and final levels of Phenylalanine as detected by the sensors were benchmarked using a GC-MS analytical method. The Final/Initial was compared to reflect the phenylalanine consumption from day 0 (FIG. 51) with similar ratios observed with the two different analytical procedures.

FIG. 33 compares the cell microscopy image over the measurement 4 days between the cell well with sensor and the cell well without sensor. Optical microscopy was performed using an Olympus IX53 inverted microscope equipped with a 10× objective. The image was directly taken from middle of each well in 24 well plate. Imaging was conducted at room temperature. There are no obvious differences on cell morphology and density between the two conditions which validate the sensor's great biocompatibility and non-toxicity for long-term experiments.

After exploring best scanning frequency in human plasma like conditions, more reliable and accurate real-time measurement results in cell culture under human plasma like conditions were achieved.

### Example 17: Agarose Gel Coating to Improve Sensor Anti-Biofouling Capabilities

The use of an agarose hydrogel protective layer for electrochemical aptamer based sensors enhances signalling stability. Gel-protected sensors demonstrate significantly improved stability, enabling precise and continuous molecular measurement in vivo (Li, S et al., 2023, ACS Nano 17 (18): 18525-38). After fabrication of the sensor gold working electrode, the agarose coating was followed by the following process (FIG. 52):
1. Dissolved agarose powder in 1×PBS at almost boiling temperature. (1.5 wt %)
2. Put the agarose solution into 60° C. for an hour.
3. Then gently dipped the sensors into the agarose solution 3 to 5 times with each immersion lasting ~3 s and stored sensors in PBS buffer prior to use.

Four phenylalanine working electrodes were fabricated, three sensors were coated with agarose gel and one was not. 50 groups of SWV scans (300 Hz and 10 Hz) were completed in working buffer without phenylalanine for each electrode. Then Phenylalanine in working buffer was titrated from 1 nM to 3 mM, and at each concentration, one group of SWV scans were completed for each electrode.

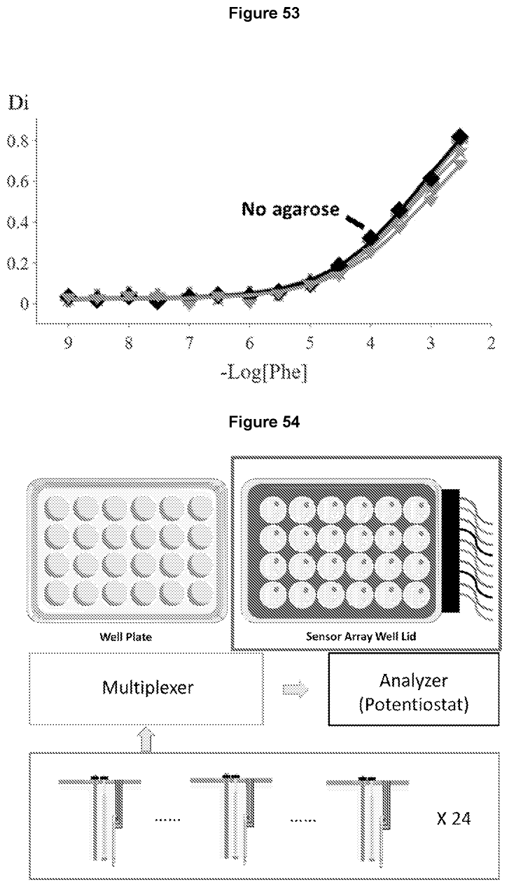

Agarose coating did not influence the aptamer phenylalanine measurement ability (FIG. 53).

### Example 18: Multiplexed Sensor Array System Design

The proposed design is shown in FIG. 54. The array was designed to be compatible for 24-well culture plate multiplexing monitoring. The sensor was embedded on the culture plate lid, each well has its corresponding sensor element. To enable the sensor array multiplex reading, the whole lid board would be suitable for connecting to a bus connector, then may be connected to a multiplexer and 8 channel potentiostat to enable reading of 24-well culture plate with one potentiostat in real-time.

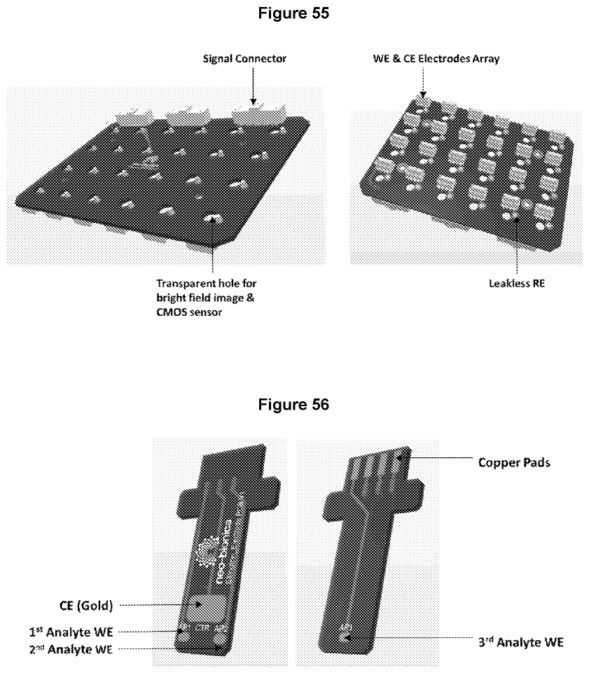

The finalized printed circuit board (PCB) is shown in FIG. 55, the board size is identical to the size of the 24-well plate, and a hole was opened on the location of each well for the bright field image and CMOS sensor integration. On one long edge of the PCB, three connectors were embedded for multiplexer connections, each was connected to two columns of sensors (8 wells). The PCB was aligned to each well and equipped with working and counter electrode arrays, with one small hole for the leakless reference electrode connection.

The sensor electrode array PCB design is shown in FIG. 56. A 3 mm*6 mm area of gold was printed on the first side of the PCB which worked as counter electrode. Two 1 mm*1 mm gold working electrodes were printed on the first side, one 1 mm*1 mm gold working electrode was printed on the second side. All four electrodes were connected to copper track. And the copper tracks were covered with insulated layer with only connection end exposed for the electronic signal transmission to the PCB.

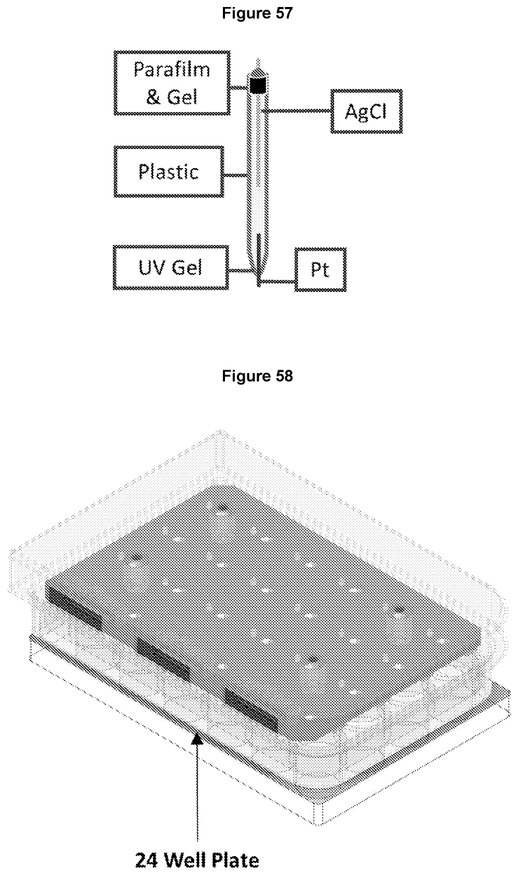

An alternative version of the reference electrode (V2) is shown in FIG. 57. The previous glass capillary tube (V1) was replaced by plastic capillary tube (V2) to improve reference electrode robustness. One end of the plastic capillary tube end was sealed by platinum wire by pinching one end of the and then fully sealed it by biocompatible UV gel. Then the plastic capillary tube was filled with 3M NaCl, inserted Ag/AgCl wire, and sealed by parafilm & gel from the other end.

The 3D printed sensor array lid design is shown in FIG. 58. The lid of the sensor array system was customized to match the size of the 24-well cell culture plate. The inner size was customized for the PCB assembly. On one side of the 3D-printed lid, three holes were left for the multiplexer and potentiostat connections.

### Example 19: Multiplexed Sensor Array System Testing

The sensor array system was fabricated and assembled as shown in FIG. 59.

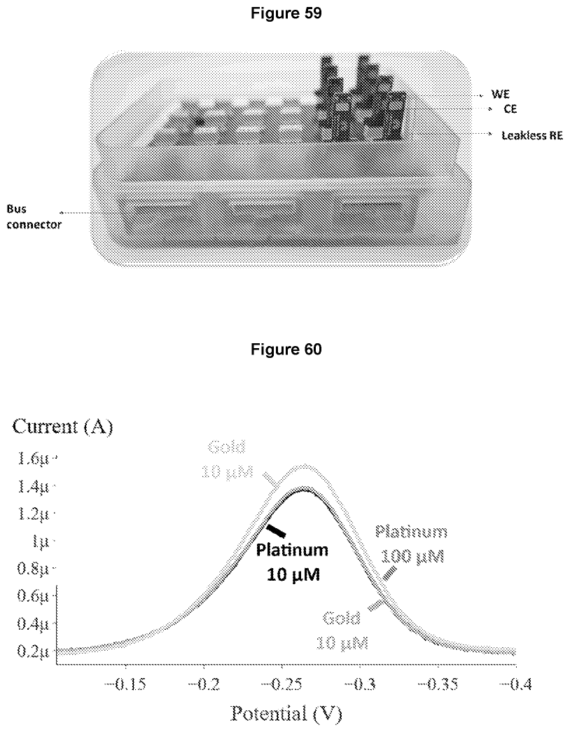

To ensure the gold counter electrode has the same measurement ability as platinum counter electrode, sensors were fabricated where gold was used as the counter electrode in place of the platinum electrode. Test sensor response curves in working buffer without phenylalanine and with phenylalanine (FIG. 60) showed that use of gold as the counter electrode did not influence the sensor measurement.

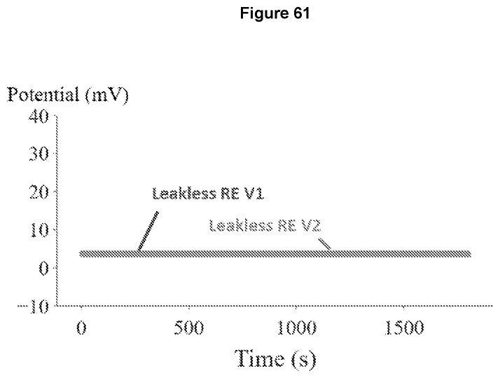

Potential stability of the plastic leakless reference electrode version 2 (plastic capillary tube) was tested against commercial reference electrode in 3M NaCl. The comparison results of two versions of the leakless reference electrodes are shown in FIG. 61. The leakless reference electrode version 2 potentials are relatively stable against commercial reference electrodes. In summary, the multiplexed sensor system provides one or more of the following advantages:
1. Multiplexed and high-throughput monitoring abilities: The multiplexed sensor system enables monitoring of multiple analytes based on the number of gold working electrodes printed on the PCB. Each working electrode can perform dual-purpose monitoring of pH and aptamer target analyte.
2. Non-cytotoxicity: The integration of leakless reference electrode prevented the leaking of silver ion from NaCl and AgCl container to the cell culture medium.
3 New analytes introduced into cell culture monitoring: Aptamer sequence may be customized to detect a wide range of target analytes including phenylalanine, tryptophan, and doxorubicin.

1. Use of an electrochemical aptamer-based sensor to detect or measure pH and a target ligand level in a solution or suspension.

2. Use according to claim 1, wherein the sensor comprises an aptamer functionalised working electrode.

3. Use according to claim 2, wherein a surface of the working electrode is functionalised with a self-assembled monolayer of a single type of aptamer.

4. Use according to claim 2 or 3, wherein the aptamer comprises a redox label.

5. Use according to claim 4, wherein the redox label is selected from the group comprising: methylene blue, ferrocene, viologen, anthraquinone or any other quinones, daunomycin, organo-metallic redox labels, for example porphyrin complexes or crown ether cycles or linear ethers, ruthenium, bis-pyridine, tris-pyridine, bis-imidizole, cytochrome c, plastocyanin, and ethylenetetraacetic acid-metal complexes, or combinations thereof.

6. Use according to claim 5, wherein the redox label is methylene blue.

7. Use according to any one of claims 2 to 6, wherein the working electrode comprises an anti-fouling polymer coating.

8. Use according to any one of claims 2 to 7, wherein the sensor comprises two or more working electrodes, wherein each working electrode comprises a different type of aptamer.

9. Use according to any one of claims 1 to 8, wherein the sensor comprises a body and a reference electrode, wherein the sensor body is encased, sealed, covered, or partially covered, to provide a substantially leak-less or leak-free reference electrode.

10. Use according to any one of claims 1 to 9, wherein the sensor is adapted to fit a well of a 24 well culture plate.

11. Use according to any one of claims 1-10, wherein the pH and concentration of the target ligand in the solution or suspension is proportional to one or more electrical signals from the sensor.

12. Use according to claim 11, wherein the one or more electrical signals is selected from: peak potential, faradic current and non-faradic current.

13. Use according to claim 12, wherein the pH of the solution or suspension is measured from the peak potential electrical signal.

14. Use according to claim 12 or 13, wherein the target ligand is detected or measured from the faradic current electrical signal.

15. Use according to any one of claims 12-14, wherein the pH and target ligand is detected or measured using a single measurement.

16. Use according to claim 15, wherein the single measurement is Square-Wave-Voltammetry.

17. Use of an array comprising a plurality of sensors according to any one of claims 1 to 16, to detect or measure pH and a plurality of target ligand levels in a solution or suspension.
